(19) United States
(12) Patent Application Publication
(10) Pub. No.: US 2012/0065681 A1
(43) Pub. Date: Mar. 15, 2012

(54) METHOD, APPARATUS, SURGICAL TECHNIQUE, SURGICAL TOOLS, AND MATERIALS FOR MINIMALLY INVASIVE ENHANCED FUSION AND RESTORATION OF KINEMATICALLY PHYSIOLOGIC SPINAL MOVEMENT

(76) Inventor: Daniel John DiLorenzo, Houston, TX (US)

(21) Appl. No.: 12/879,012

(22) Filed: Sep. 10, 2010

(51) Int. Cl. A61B 17/70

(52) U.S. Cl. 606/246

(57) ABSTRACT

The present invention teaches a method and apparatus for providing and preserving kinematically correct spinal movement as well as enhanced bone fusion. The design of implanted and other devices fabricated from at least one of metal, ceramic, plastic, shape memory alloy, and other material is taught for facilitating approximately physiologic kinematics for intervertebral body movement as well as for enhanced fusion of vertebral bodies and components therein. Surgical tools and techniques for implantation of these devices made from a variety of materials are also taught.

### RELATED APPLICATIONS

This application is an original application.

### BACKGROUND OF THE INVENTION

1. Field of the Invention

The present invention relates generally to spinal surgery and, more particularly, to spinal disc replacement, including but not limited to cervical disc replacement, thoracic disc replacement, lumbar, and lumbosacral disc replacement.

Spinal surgery for degenerative discs and spinal instability has traditionally consisted of a variety of techniques designed to achieve fusion of adjacent spinal vertebra. This is generally accomplished via anterior, lateral, or posterior approaches. A spectrum of procedures, surgical tools, and implanted devices have been devised to facilitate fusion by a multitude of permutations of these various approaches.

Fusion is generally used for treating axial or radicular pain, neurological deficits, and mechanical instability. Underlying causes of these conditions usually include excessive or abnormal motion between adjacent vertebral bodies, motion involving degenerative vertebral body endplates and/or facets, abnormal motion due to fracture, or abnormal motion due tear or strain of ligaments, tendons, or disc annulus. Pain and neurological deficits may also be caused by hyperostosis or herniated or protruding disc; and, depending on anatomy and patient health and condition, these also may be treated with fusion or with decompressive surgery.

Though often transiently successful for several years, fusion has significant drawbacks. First, spinal motion is inherently reduced. Depending on the involved spinal level, this may be barely noticeable or may impose significant limitations in movement, particularly if involving the cervical spine. Second, due to the restricted motion as the fused segments, abnormal and incrementally more motion occurs at adjacent spinal levels; this is believed to play a role in adjacent disc disease, in which discs adjacent to fussed segments degenerate at a faster rate and often require fusion at a later time.

More recently, arthroplasty has been introduced as a potential solution to the two problems discussed above posed by spinal fusion. Arthroplasty is defined as “an operative procedure of orthopedic surgery performed, in which the arthritic or dysfunctional joint surface is replaced with something better or by remodeling or realigning the joint by osteotomy or some other procedure.” (Wikipedia Feb. 23, 2010). Note the mention of the “joint surface”; the reconstruction of a joint surface has been the staple approach fro joint arthroplasty. To the knowledge of the inventor, all artificial spinal discs have employed an approach dependent upon the creation of at least one and generally a pair of artificial surfaces which glide relative to each other to provide an approximation for normal joint movement.

Though some devices provide limited curvature of the surfaces, the actual movement of the device is along or tangential to a 2-dimensional surface with no movement orthogonal to this surface. As a result, these earlier designs force adjacent vertebral bodies to move in directions which are approximately tangential to the axial plane. This is inconsistent with the natural movement of the facet joints, which primarily provide rotation. Consequently, artificial discs employing sliding surfaces place the facet joints under abnormal stress and strain conditions and are likely to accelerate wear and tear on these joints, thereby causing postoperative pain, enhanced degeneration of facet joints at the implanted level and potentially accelerated degeneration of discs and facets at adjacent levels, a problem for which artificial discs were introduced to overcome in the first place!

Furthermore, artificial surfaces which more relative to each other convey the risk of microparticle generation and diffusion into adjacent and distant tissues. This can incite an inflammatory response, which can potentially have severe long-term consequences or sequalae.

Additionally, inherent in a design which employs sliding rigid surfaces is an absence of significant mechanical compliance. This causes reduction in the ability of the spinal column to absorb impact and dissipate energy from forces with dynamic components, such as many forces endured during daily life, such as those from walking, running, exercising, and other normal or stressful activities.

2. Related Art

For the correction of spinal instability, advanced degenerative disc disease, and other conditions, previous spinal fusion techniques and technologies have typically relied upon the implantation of at least one of (1) an intervertebral element (cadaveric bone, artificial or synthetic material, metal cage), and (2) posterior segmental instrumentation (typically rods and screws), each of which are designed to provide mechanical support while adjacent vertebral bodies fused.

Some segmental stabilization devices, most notably anterior cervical plates, have employed “dynamic” designs, facilitate movement which allows subsidence, or settling of the adjacent vertebral bodies, while preserving the integrity of the plate and its fixation to the bone using screws. These “dynamic” systems generally facilitate pure translational movement by (A) employing a plate with slots, through which screws translate or slide as the vertebral bodies move towards each other as the intervertebral space collapses, or (B) employing plates facilitating variable angle screws, which allows the screws to change angle as the vertebral bodies move towards each other in a translational manner. These dynamic systems are passive, with often unpredictable rates of movement (or “subsidence”), with varying and unpredictable forces across the fusion surface. There forces and movements are uncontrolled and subject to external influences and as such change with the movements, position (recumbency versus standing posture) of a person, and other uncontrolled factors.

In an attempt to preserve some intervertebral segment movement, artificial discs have been produced which facilitate limited and restricted movement. Currently available devices do not preserve nor reproduce the normal physiologic kinematics of the intervertebral joints nor intervertebral segment movements. Because of this non-physiologic movements, progressive or accelerated damage to the often already degenerated facet joints ensues, and a significant portion of artificial discs implanted for the purpose of motion segment preservation ultimately result in fusion of that segment following implantation of the artificial disc.

### SUMMARY OF THE INVENTION

The present invention teaches apparatus and methods for treating a multiplicity of diseases, including but not limited to spinal degenerative disc disease, spinal stenosis, myelopathy, myelomalacia, foramenal stenosis, radiculopathy, spinal instability, spondylolisthesis, anterolisthesis, retrolisthesis, spinal fracture, vertebral body fracture, chance fracture, jumped facet, facet fracture, pars fracture, neural arch fracture, other disorder involving the spine, including the craniocervial, cervical, cervicothoracic, thoracic, thoracolumbar, lumbar, lumbosacral, sacral, sacrococcygeal, coccyx regions of the spine, fractures of other bones including but not limited to those of the upper and lower extremities, calvarum and maxillofacial bones, pelvis, axial skeleton, other bones, and other diseases. The present invention also teaches surgical techniques and tools for placing these devices, neurostimulation systems, neural interfaces, and other devices, using at least one of open surgical technique, minimally invasive surgical technique, percutaneous technique, or other technique.

The invention taught herein employs a variety of implanted devices, other devices, surgical techniques, and surgical tools, to deliver the treatments. These techniques include open surgical techniques as well as minimally invasive surgical techniques, including percutaneous application of devices.

Implanted devices comprise (1) vertebral cages, for the restoration of intervertebral body space and for mechanical stabilization of vertebral bodies during fusion; (2) artificial intervertebral discs (for arthroplasty surgical procedures and minimally invasive procedures) for restoration and maintenance of intervertebral kinematics and dynamics, including normal physiologic kinematics and dynamics, (3) dynamic loaded fusion compression systems (surgically implanted, microsurgically implanted, minimally invasively placed, noninvasive) for a variety of application comprising fracture (including those listed above in this section), intervertebral body fusion, other fusion, mechanical stabilization, and other uses; (4) dynamic loaded segmental instrumentation (including that with loading applied from the at the rod, attachment, pedicle screw, facet screw, laminar screw, other screw, or other element) which applies controlled compression with at least one of translational and rotational force in any of the 6 degrees of freedom for each motion segment; and (5) impedance matched segmental instrumentation, which employs designs which provide mechanical impedances which may vary to match the varying mechanical impedances of the involved levels (including the provision of impedance transitions across central and terminal regions as well as across fused, mobile, normal, and terminal levels within and beyond the range of the instrumented levels.

Surgical tools comprise: (6) surgical tools for minimally invasive and minimally traumatic surgical procedures, including those for preparation of the disc space; and (7) implantation of intervertebral devices, including fusion cages, artificial discs, and dynamic loaded instrumentation and other instrumentation for fusion and other procedures.

### INCORPORATION BY REFERENCE

All publications, patents, and patent applications mentioned in this specification are herein incorporated by reference to the same extent as if each individual reference, publication, patent, or patent application was specifically and individually indicated to be incorporated by reference.

Furthermore U.S. Provisional Application 60/307,124, filed Jul. 19, 2001 is incorporated by reference, and all patents claiming priority to this application are also incorporated by reference.

U.S. application Ser. No. 10/198,871, now U.S. Pat. No. 7,599,736 (Docket GISTIM 01.01), filed Jul. 19, 2002 and issued Oct. 6, 2009 and all patent applications and patents claiming priority to this application are incorporated by reference.

U.S. application Ser. No. 10/872,549, now U.S. Pat. No. 7,529,582 (Docket GISTIM 01.02), filed Jun. 21, 2004 and issued May 5, 2009 and all patent applications and patents claiming priority to this application are incorporated by reference.

U.S. application Ser. No. 11/317,099 (Docket GISTIM 02.02), filed Dec. 22, 2005 and all patent applications and patents claiming priority to this application are incorporated by reference.

### BRIEF DESCRIPTION OF DRAWINGS

FIG. 1 depicts an intervertebral dynamic cage, with an elastic cage frame and a viscoelastic element for dampening.

FIG. 2 is an intervertebral dynamic cage with elastic segment tabs and slots for controlling elasticity and rigidity of anterior and posterior elastic segments. This possesses curved elastic elements and may be fabricated from a single piece or multiplicity of pieces of material.

FIG. 3 is an intervertebral dynamic cage with elastic segment tabs and slots for controlling elasticity and rigidity of anterior and posterior elastic segments. This possesses multiple-curved elastic elements and may be fabricated from a single piece or multiplicity of pieces of material.

FIG. 4 is an intervertebral dynamic cage with elastic segment tabs and slots for controlling elasticity and rigidity of anterior and posterior elastic segments. This possesses coiled elastic elements and may be fabricated from a single piece or multiplicity of pieces of material.

FIG. 5 is a 3-dimensional depiction of an expandable artificial disc with a multi-curved spring element. This possesses coiled elastic elements and may be fabricated from a single piece or multiplicity of pieces of material.

FIG. 6 is an intervertebral dynamic cage with elastic segment tabs and slots for controlling elasticity and rigidity of the posterior elastic segment. This possesses multiple-coiled elastic elements and may be fabricated from a single piece or multiplicity of pieces of material.

FIG. 7 is an intervertebral dynamic cage with elastic segment tabs and slots for controlling elasticity and rigidity of anterior and posterior elastic segments, which may have differing elasticities. This possesses multiple-curved elastic elements and may be fabricated from a single piece or multiplicity of pieces of material.

FIG. 8 is a 3-dimensional depiction of an expandable artificial disc with a coiled spring element. This possesses coiled elastic elements and may be fabricated from a single piece or multiplicity of pieces of material.

FIG. 9 is an anterior, lateral, posterior, or oblique view of a dynamic loaded intervertebral artificial disc in various configurations before and after insertion into the intervertebral space; this may be placed via any approach at any level including occipitocervical, cervical, thoracic, lumbar, sacral, sacrococcygeal levels.

FIG. 10 is diagram relating the temperature and phase of a shape memory material, including a shape memory alloy.

FIG. 11 is an anterior, lateral, posterior, or oblique view of a dynamic loaded intervertebral artificial disc in various configurations before and after insertion into the intervertebral space; this may be placed via any approach at any level including occipitocervical, cervical, thoracic, lumbar, sacral, sacrococcygeal levels.

FIG. 12 is an anterior, lateral, posterior, or oblique view of a dynamic loaded plate and a dynamic loaded intervertebral artificial disc in various configurations before and after insertion into the intervertebral space; this may be placed via any approach at any level including occipitocervical, cervical, thoracic, lumbar, sacral, sacrococcygeal levels.

FIG. 13 is an anterior, lateral, posterior, or oblique view of a dynamic loaded plate implanted on two vertebral bodies with an intervening artificial intervertebral disc and a series of diagrams depicting the sequence of steps and plate configurations comprising the implantation procedure. This may be placed via any approach at any level including occipitocervical, cervical, thoracic, lumbar, sacral, sacrococcygeal levels.

FIG. 14 is an anterior, lateral, posterior, or oblique view of a dynamic loaded plate implanted on two vertebral bodies with an intervening intervertebral fusion graft and a series of diagrams depicting the sequence of steps and plate configurations comprising the implantation procedure. This may be placed via any approach at any level including occipitocervical, cervical, thoracic, lumbar, sacral, sacrococcygeal levels.

### DETAILED DESCRIPTION OF THE INVENTION

Applications:

The present invention encompasses a multimodality technique, apparatus, and method, for the treatment of a multiplicity of diseases, including but not limited to spinal degenerative disc disease, spinal stenosis, myelopathy, myelomalacia, foramenal stenosis, radiculopathy, spinal instability, spondylolisthesis, anterolisthesis, retrolisthesis, spinal fracture, vertebral body fracture, chance fracture, jumped facet, facet fracture, pars fracture, neural arch fracture, other disorder involving the spine, including the craniocervial, cervical, cervicothoracic, thoracic, thoracolumbar, lumbar, lumbosacral, sacral, sacrococcygeal, coccyx regions of the spine, fractures of other bones including but not limited to those of the upper and lower extremities, calvarum and maxillofacial bones, pelvis, axial skeleton, other bones, and other diseases. The present invention also teaches surgical techniques and tools for placing these devices, neurostimulation systems, neural interfaces, and other devices, using at least one of open surgical technique, minimally invasive surgical technique, percutaneous technique, or other technique.

Objectives:

One objective of the present invention is to provide a more physiologic and more kinematically correct movement than joint surfaces are capable of providing. Joint surfaces inherently provide motion in 2 dimensions

Another object of the present invention is the creation of an artificial disc which is constructed substantially from a single piece or from a construct which functions as a single piece. This may be implemented by any of several apparati, including a single or multiplicity of metal pieces, a single or multiplicity of nonmetal pieces, a single or multiplicity of plastic pieces, or a single or multiplicity of pieces constructed from similar or dissimilar materials. This may be implemented as a construct fashioned from multiple pieces which are welded, adhered, glued, affixed, bonded, screwed, fastened, secured, mechanically secured, or otherwise rendered in mechanical communication. The said multiple pieces may be bonded using electrical energy, optical energy, vibratory energy, ultrasonic energy, mechanical energy, mechanical force, chemical means, biological means, or other technique or method for affixing, mechanically attaching, or rendering relatively immobile or connected with restricted relative motion.

Yet another object of the present invention is the facilitation of artificial disc implantation via a relatively minimally invasive and minimally traumatic surgical procedure. One preferred method for accomplishing this novel objective is the use of an expanding disc material. This may be triggered by one or more mechanisms, including mechanical, electrical, thermal, optical, or other mechanism. Expansion may be activated upon implantation within the body; one such novel method employs the use of Nickel-Titanium alloy (nitinol) material, which may change shape upon temperature change. The device may be in a collapsed state at room temperature, facilitating implantation via a minimally invasive technique and incision. Upon contact with the body, the material may change shape and attempt to re-expand to its expanded dimensions, resulting in a secure fit within the disc space between the adjacent vertebral bodies. Using an expanding device, as taught herein, facilitates novel less invasive procedures and minimally invasive procedures for the implantation or placement of such a device.

Another object of the present invention is the facilitation of superior surgical and other techniques using novel devices, instrumentation, and surgical tools to create fusion and nonfusion treatments for spinal disease. One such feature involves impedance matching, in which the mechanical impedance of a device, such as a posterior instrumentation rod, varies along the length of the device to match the impedance of a fusion segment with the impedances of nonfusion segments, thereby reducing the otherwise elevated stress and strain at the adjacent nonfusion segments. One application for this is the reduction of adjacent level disease. Impedance matching may also be used to protect segment which have undergone arthroplasty by providing some augmented mechanical support, such as compression or such as the application of various impedances to movement in one or a multiplicity of dimensions. This may provide benefit in the form of load sharing for extending the lifespan of the artificial disc as well as the provision of more physiologic movement, to reduce wear on other structures, such as facet joints, ligamentum flavum, other ligaments and structures, at that level as well as to these structures and discs, artificial discs, and fusion constructs at adjacent or distant levels. These technologies have application for single and multiple segment procedures. One such application is that which involves fusion at one or more levels, flanked with artificial discs at adjacent levels, and nonfusion at subsequent adjacent levels.

A further object of the present invention is the provision of enhanced fusion rates in spinal fusion procedures through the use of at least one of dynamic loading, constrained dynamic fixation, and constrained dynamic loading. This technique and associated devices, methods, and surgical tools us any of several approaches to improve fusion rates and performance. This may be applied to vertebral body fusion, posterior element fusion, or fusion of any other component or aspect of spinal bone or support structures, including occipital, occipitocervical, cervical, cervicothoracic, thoracic, thoracolumbar, lumbar, lumbosacral, sacral, and sacrococcygeal bones. This may also be applied to the fusion of other bones, including the calvarum, craniofacial bones, upper limb bones, lower limb bones, pelvic bones, axial skeletal bones, appendicular skeletal bones, and other bones.

One objective of the present invention is the restoration and maintenance of kinematically correct movement between adjacent and distant vertebral bodies. The physiologically normal intervertebral disc facilitates movement in multiple axes and shared load bearing with facet joints. When disc degeneration occurs, movement kinematics are compromised, and physiologically abnormal forces and movements (producing abnormal pressures and strains) are applied to the facet joints, various ligaments, and disc capsules. These abnormal forces and movements produce abnormal wear on these structures, and physiological compensatory mechanism result in hypertrophy of these structures as well as combination of helpful and detrimental adaptations. Hypertrophy of the facets, ligaments (especially the ligamentum flavum), and vertebral body bone at the bone-disc interface act to reinforce the spinal structures to reduce abnormal movements. While potentially helpful in this kinematics respect, the increased dimensions of these solid structures comes at the expense o decreased dimensions of the complementary adjacent potential spaces, specifically the spinal canal and the neural foramina. Two significant detrimental consequence of these reductions in potential space are spinal stenosis and foramenal stenosis, respectively. These may result in significant patient morbidity, including myelopathy (die to compression of and damage to the spinal cord) and radiculopathy (due to compression of and damage to the spinal nerve).

One objective of the Kinematically physiologic artificial disc (for use in the Kinematically Physiologic Arthroplasty procedure) is the restoration of physiological movement between spinal segments. The benefits of this feature are multifold.

Ideals:

1. Restoration of the normal disc height will decompress neural foramina and may reduce compression of the spinal canal by acting to stretch the ligamentum flavum, intervertebral disc, and disc capsule.

2. Restoration of the normal physiologic kinematics will reduce or eliminate the pathological loads which are a root cause for the pathologic processes in which hypertrophy of these various structures results in neural compression as well as malfunction and further degeneration of the structures themselves.

3. Restoration of the normal physiologic kinematics will reduce or eliminate the progressive or accelerated degenerative processes which concurrently occur at adjacent disc levels.

Fundamental Improvements Over Existing Technologies:

A. Fusion: One problem with fusion is the potential for increased force/pressure loads and strains at adjacent disc levels, attributed for the “adjacent level disease” felt to be accelerated following fusion. Maintenance of physiologic kinematics will significantly reduce or eliminate this problem, which is felt to be a major cause of patient morbidity and downstream costs to the healthcare system.

B. Artificial Discs: Present artificial discs provide some movement at the intervertebral space; however, this is typically not physiologic. The achieved movement is constrained by the artificial disc design. Much of the movement is constrained to a plane approximately parallel to the endplate surface; this does not permit vertical translational movement, which is responsible for physiologic movement within the sagittal plane (bending the back forward and extending the back backwards). By restricting movement to such a plane, it is anticipated that wear and tear of The remaining facet joints will be accelerated, resulting in the potential for accelerated neural foramenal stenosis, neural canal stenosis, and ultimate fusion of the arthroplasty as the joint degeneration progresses.

Minimally Invasive Surgical Technique

The apparatus and method taught in the present case is enabling to an engineer or engineering team skilled it the art of developing spinal instrumentation and craniofacial instrumentation systems, and the anatomical placement of devices is enabling to a competent clinical practitioner, who has an armamentarium of surgical implantation techniques at his or her disposal. For sake of completeness, example apparatus and procedures are presented in the present application.

Apparatus and methods are taught herein which facilitate the collapse of an implanted intervertebral artificial disc and/or plate, facilitating implantation using a smaller incision that is required using present technologies.

Additionally, apparatus and methods are taught herein which facilitate the collapse of an implanted intervertebral artificial disc and/or plate, thereby obviating the need to substantially enlarge the height of collapsed intervertebral disc height, potentially reducing the scope and angle of tool access and exposure required to perform the implantation procedure.

Furthermore, apparatus and methods are taught herein which facilitate the enhanced rates of fusion and enhanced rates of bone intercalation with implant material, potentially facilitating the use of smaller plates and instrumentation since more rapidly fused bone may assume load bearing more quickly and reduce the duration for which implants are expected to bear the load of fusing bones.

### DETAILED DESCRIPTION OF THE DRAWINGS

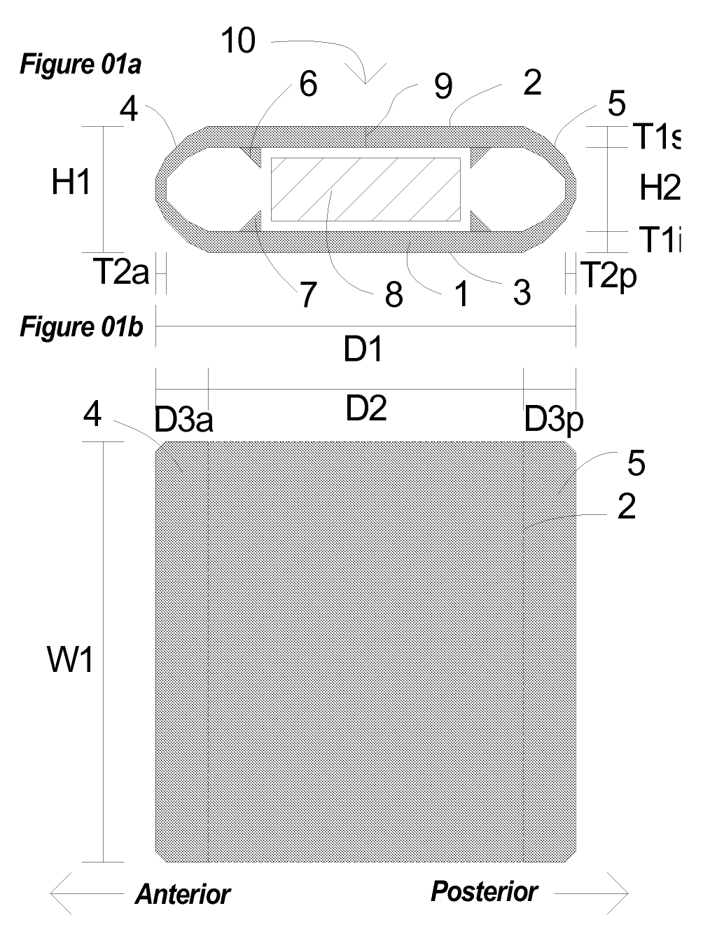

FIG. 1 depicts a dynamic loaded intervertebral artificial disc 10. Dynamic loaded element 1 comprises superior endplate articulating surface 2 and inferior endplate articulating surface 3, which are configured to be in mechanical communication with inferior endplate of adjacent superior vertebral body and with superior endplate of adjacent inferior vertebral body, respectively.

This dynamic loaded intervertebral artificial disc 10 may comprise a single or multiple piece intervertebral dynamic cage, with an elastic single or multiple piece cage frame and a viscoelastic element for dampening

Dynamic loaded element 1 may be constructed from a single-piece or a multiplicity of pieces. Dynamic loaded intervertebral artificial disc 10 may be constructed from a single-piece or a multiplicity of pieces. Anterior elastic element 4 and posterior elastic element 5 may be fabricated from the same piece of material from which dynamic loaded element 1 is fabricated. Superior surface insert restraint 6 and Inferior surface insert restraint 7 are mechanically attached to or formed as a single piece and part of dynamic loaded element 1, and they serve to mechanically constrain and secure viscoelastic insert block 8.

Viscoelastic insert block 8 may be embodied in several forms without departing from the present invention. Viscoelastic insert block 8 may be a homogeneous viscoelastic mechanical element, in homogeneous viscoelastic mechanical element, network of viscoelastic mechanical elements, homogeneous viscous mechanical element, in homogeneous viscous mechanical element, network of viscous mechanical elements, homogeneous elastic mechanical element, in homogeneous elastic mechanical element, network of elastic mechanical elements, or component of other mechanical properties.

In one preferred embodiment, dynamic loaded element 1 is placed in a state of compression at least one of prior to and during insertion or implantation. This may be accomplished through the use of a compressing tool used during implantation or through temperature control, in which case dynamic loaded element 1 is fabricated from a shape memory alloy such as a Nickel-Titanium alloy (such as approximately 50% Nickel and 50% Titanium, though departures form this ration are also included in the present invention). Upon placement in the intervertebral space, at least one of the compressive force is released and the temperature is changed, allowing the dynamic loaded element 1 to expand into the intervertebral space, placing the dynamic loaded element 1 and adjacent vertebral body endplates and vertebral bodies in compression. This will encourage new bone formation in the region of the implant, as held by Wolff's Law, and further increase the mechanical stability of the artificial disc to vertebral body junction.

Dynamic loaded element 1 may be constructed as a single piece through manufacturing techniques employing extrusion or other method for producing a single weld-free piece. Alternately, dynamic loaded element 1 may be constructed in an open shape and adhered to form a closed shape as depicted using thermal welding, spot welding, electrical welding, adhesive boning, mechanical linking, mechanical interlocking, dove tail locking, or other method for attaching the ends to form a junction 9.

Dynamic loaded element 1 may be constructed from at least one of stainless steel, titanium, polyetheretherketone, nickel-titanium alloy, other shape memory alloy, and other material. Viscoelastic insert block 8 may be constructed from at least one of silicone, Teflon, polyetheretherketone, and other material.

Dimensions shown include outer height H1, inner height H2, outer depth D1, inner depth D2, anterior elastic segment depth D3a, posterior elastic segment depth D3p, superior articulating surface plate thickness T1s, inferior articulating surface plate thickness T1i, anterior elastic member thickness T2a, and posterior elastic member thickness T2p.

Alternatively, one of anterior elastic element 4 and posterior elastic element 5 may be omitted, producing a “C” shaped cross section with a cantilever mechanical behavior, with potentially less rigidity and simpler stress distribution than the closed “O” cross section design. Alternatively, both anterior elastic elements 4 and posterior elastic element 5 may be omitted, producing an “H” or “II” shaped cross section with mechanical behavior dependent upon the central viscoelastic insert block 8, with potentially less dynamic loading and less elastic force and more viscous damping, depending on the properties of the viscoelastic insert block 8 used.

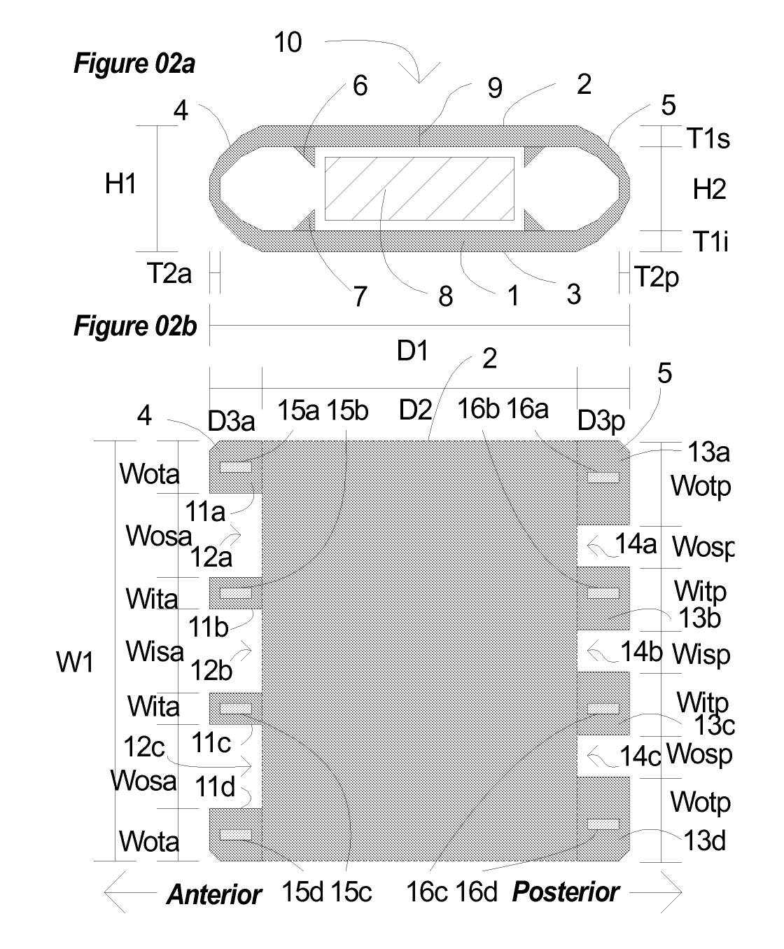

FIGS. 2, 3, 4, 6, and 7 depict a dynamic loaded intervertebral artificial disc 10, as shown in FIG. 1 with the addition of elastic segment tabs 11 (shown as 11s through 11d) and 13 (shown as 13a through 13d), elastic segment slots 12 (shown as 12a through 12c) and 14 (shown as 14a through 14c), and elastic segment sensors 15 (shown as an array 15a through 15d) and 16 (shown as an array 16a through 16d). Dynamic loaded intervertebral artificial disc 10 may be constructed from a single-piece or a multiplicity of pieces. These additional elements facilitate the specific tailoring of elastic properties of the anterior elastic element 4 and posterior elastic element 5, which may have the same mechanical properties or different mechanical properties. By appropriately designing the elastic segment tabs 11 and 13, elastic segment slots 12 and 14, and thicknesses T2a and T2b, the mechanical properties of anterior elastic segment 4 and posterior elastic segment 5, respectively, can be controlled. One preferred embodiment specifies identical mechanical properties for anterior elastic segment 4 and posterior elastic segment 5. Another preferred embodiment specifies differing mechanical properties for anterior elastic segment 4 and posterior elastic segment 5.

Another specific preferred embodiment specifies greater elasticity (less rigidity, i.e. a lower spring constant) for the anterior elastic segment 4 in comparison to the posterior elastic segment 5. This design will place the effective axis of rotation for the artificial disc and adjacent vertebral bodies closer to the posterior elastic segment 5 than the anterior elastic segment 4. This will allow the postoperative axis of rotation to be more easily set in the posterior half of the vertebral interspace, more accurately recreating physiologically correct spinal kinematics.

Dynamic loaded element 1 may be constructed from at least one of stainless steel, titanium, polyetheretherketone, nickel-titanium alloy, other shape memory alloy, and other material. Viscoelastic insert block 8 may be constructed from at least one of silicone, Teflon, polyetheretherketone, and other material.

FIG. 2 depicts a design in which the anterior elastic segment 4 and posterior elastic segment 5 are curved, including but not limited to a hemi-circular curve, and arc curve, an oval curve, a parabolic curve, other curve.

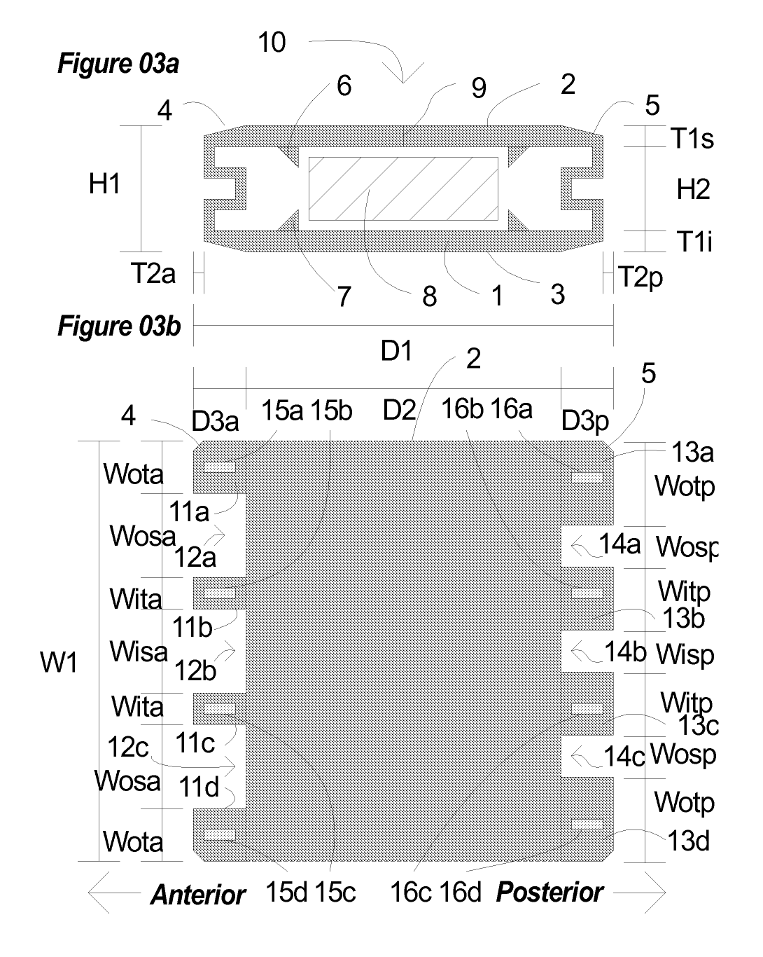

FIG. 3 depicts a design in which the anterior elastic segment 4 and posterior elastic segment 5 have a multiple curved or angled segments, including but not limited to at least one of a hemi-circular curve, arced curve, oval curve, parabolic curve, rectangular angle, acute angle, obtuse angle, other curve, other angle, combination of at least one curve, combination of at least one angle, combination of at least one curve and at least one angle, or other shape

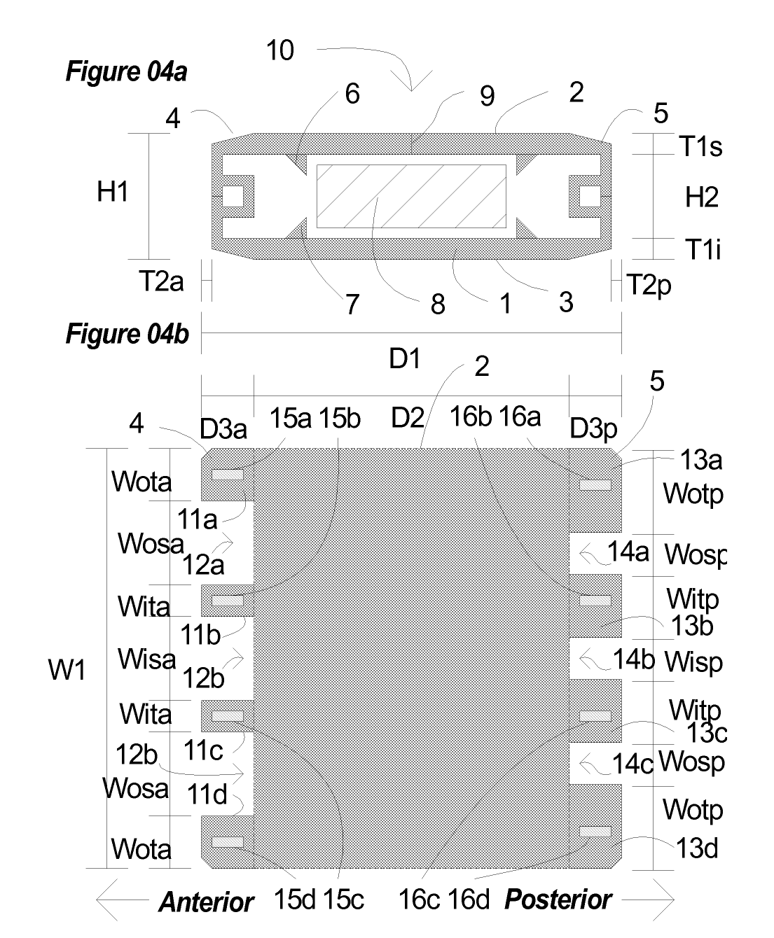

FIG. 4 depicts a design in which the anterior elastic segment 4 and posterior elastic segment 5 are coiled, including but not limited to a circular curve, multi-loop circular curve, arc curve, a multi-loop arced curve, an oval curve, a multi-loop oval curve, a parabolic curve, a multi-loop parabolic curve, other curve, or other multi-loop curve.

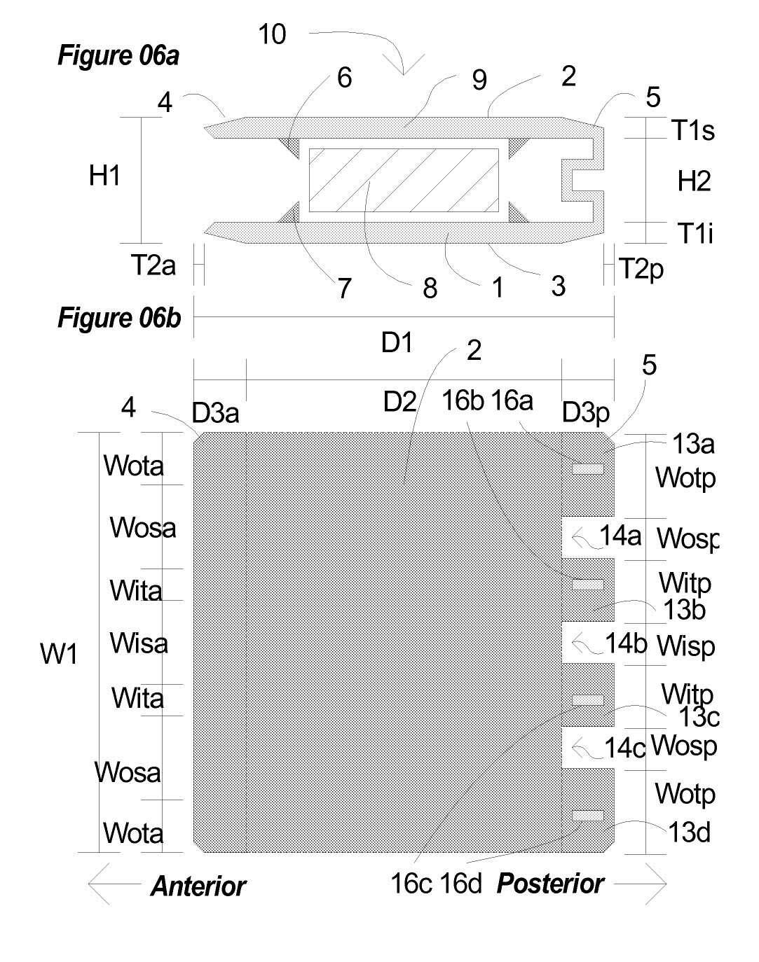

FIG. 6 depicts a design in which the anterior elastic segment 4 has minimal rigidity or is absent and posterior elastic segment 5 comprises a curved, multiple curved, angled, multiple angled, coiled, multiple coiled design including but not limited to a circular curve, multi-loop circular curve, arc curve, a multi-loop arced curve, an oval curve, a multi-loop oval curve, a parabolic curve, a multi-loop parabolic curve, other curve, or other multi-loop curve.

This may be fabricated from a single piece of material or a multiplicity of pieces of material. The insert block, termed “viscoelastic insert block” may be a viscoelastic element, a viscoelastic element and a stop, or other construct. A spacer device may be placed percutaneously laterally into vertebral bodies to effect expansion of the intervertebral space.

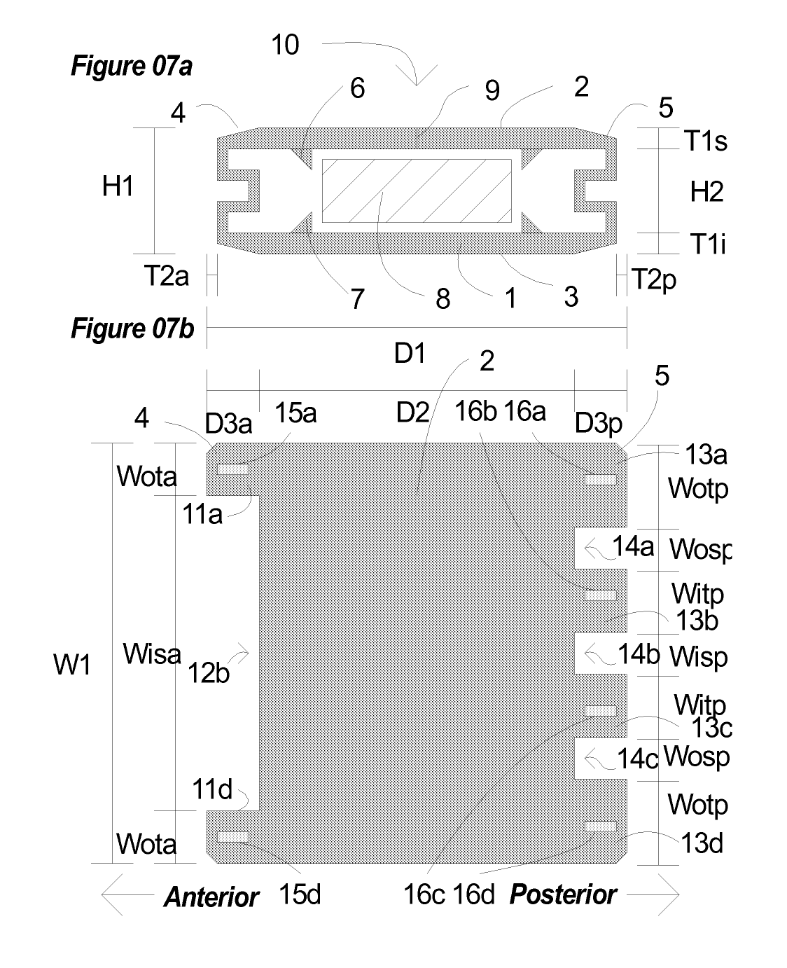

FIG. 7 depicts a design in which the anterior elastic segment 4 has at least one of no rigidity, minimal rigidity, less rigidity than posterior elastic segment 5, wider anterior elastic element slots 12, and narrower anterior elastic element tabs 11. Posterior elastic segment 5 comprises a curved, multiple curved, angled, multiple angled, coiled, multiple coiled design including but not limited to a circular curve, multi-loop circular curve, arc curve, a multi-loop arced curve, an oval curve, a multi-loop oval curve, a parabolic curve, a multi-loop parabolic curve, other curve, or other multi-loop curve.

Wide anterior elastic element slots 12 may permit placement of an element within the intervertebral space, such as but not limited to a compressible member, an elastic member, a rigid member, a fusion cage, and adjustable cage, or other device.

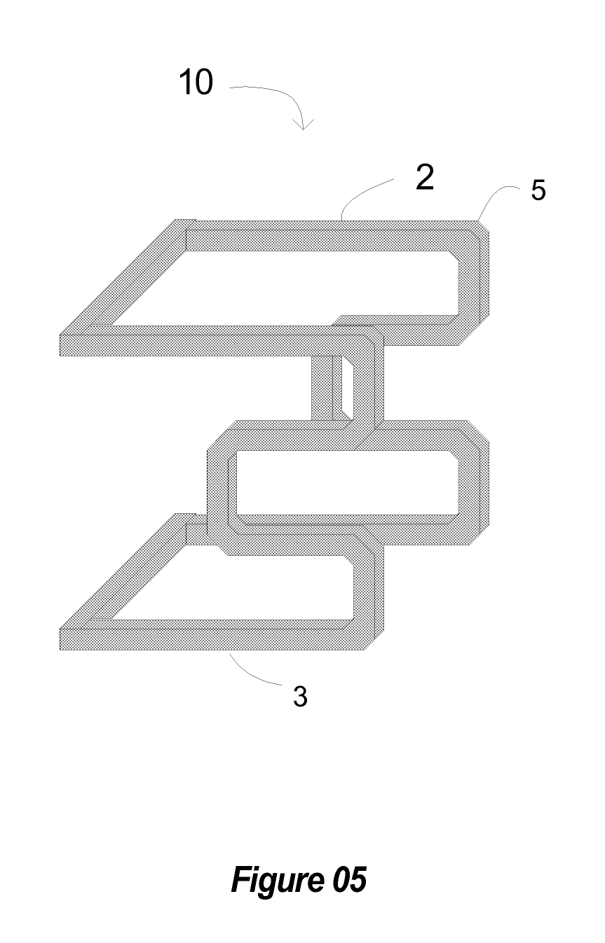

FIG. 5 depicts a dynamic loaded intervertebral artificial disc 10, which may be constructed from a single piece or a multiplicity of pieces. This embodiment of a dynamic loaded intervertebral artificial disc 10 comprises a multi-curved posterior elastic segment 5. This dynamic loaded intervertebral artificial disc 10 may be implemented as a single piece or multiplicity of pieces. Dynamic loaded element 1 comprises superior endplate articulating surface 2 and inferior endplate articulating surface 3, which are configured to be in mechanical communication with inferior endplate of adjacent superior vertebral body and with superior endplate of adjacent inferior vertebral body, respectively.

FIG. 5 depicts a design in which the anterior elastic segment 4 has minimal rigidity or is absent and posterior elastic segment 5 comprises a curved, multiple curved, angled, multiple angled, coiled, multiple coiled design including but not limited to a circular curve, multi-loop circular curve, arc curve, a multi-loop arced curve, an oval curve, a multi-loop oval curve, a parabolic curve, a multi-loop parabolic curve, other curve, or other multi-loop curve.

This dynamic loaded intervertebral artificial disc 10 may be constructed from a wire material, a drawn material, a die cast material, a forged material, or material fabricated using another process or processes.

Dynamic loaded element 1 may be constructed from at least one of stainless steel, titanium, polyetheretherketone, nickel-titanium alloy, other shape memory alloy, and other material.

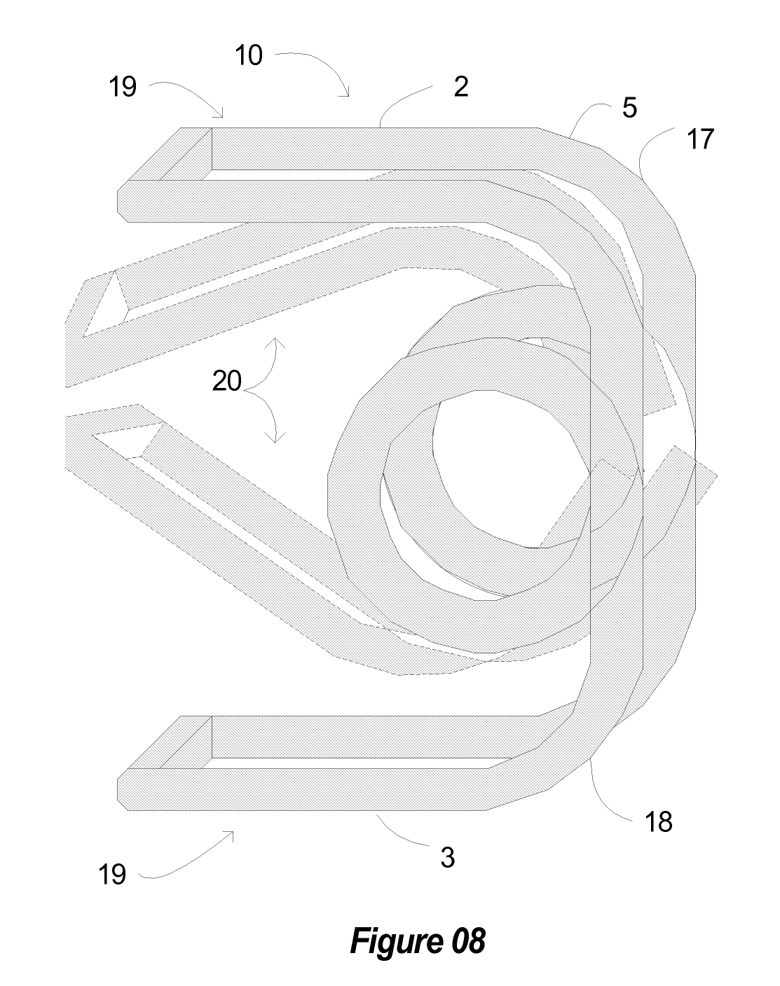

FIG. 8 depicts a dynamic loaded intervertebral artificial disc 10, as shown in FIGS. 5 and 6. This dynamic loaded intervertebral artificial disc 10 may be implemented as a single piece or multiplicity of pieces. Dynamic loaded element 1 comprises superior endplate articulating surface 2 and inferior endplate articulating surface 3, which are configured to be in mechanical communication with inferior endplate of adjacent superior vertebral body and with superior endplate of adjacent inferior vertebral body, respectively.

FIG. 8 depicts a design in which the anterior elastic segment 4 has minimal rigidity or is absent and posterior elastic segment 5 comprises a curved, multiple curved, angled, multiple angled, coiled, multiple coiled design including but not limited to a circular curve, multi-loop circular curve, arc curve, a multi-loop arced curve, an oval curve, a multi-loop oval curve, a parabolic curve, a multi-loop parabolic curve, other curve, or other multi-loop curve.

FIG. 8 depicts a design in which the posterior elastic segment 5 comprises a superior endplate articulating surface articulation recess 17 and an inferior endplate articulating surface articulation recess 18, which are novel features which effectively reduce the vertical dimension of the dynamic loaded intervertebral artificial disc 10 when in a contracted configuration 20. When in an expanded configuration 19, height of dynamic loaded intervertebral artificial disc 10 is closer to is maximum height. When in a contracted configuration 20, height of dynamic loaded intervertebral artificial disc 10 is less than its height in the expanded configuration 19. This may be used in several advantageous ways. During insertion of the device, it may be kept in a contracted configuration 20, facilitating easier implantation. After implantation and positioning, the configuration of dynamic loaded intervertebral artificial disc 10 may be changed to be in an expanded configuration 19. This can also serve several advantageous purposes:

(1) Mechanical stabilization of implant. The implant is held firmly in place in the intervertebral space by providing mechanical securing of the dynamic loaded intervertebral artificial disc 10 against the adjacent vertebral body endplates due to the tangential friction force resulting from the applied tangential force.

(2) Distracting force. Expansion of the dynamic loaded intervertebral artificial disc 10 into the expanded configuration 19 provides a compressive force at the vertebral body endplates, resulting in expansion of the intervertebral space; this correspondingly provides expansion of the disc, expansion of the neural foramen height, and potentially expansion of the spinal canal diameter.

This transition of dynamic loaded intervertebral artificial disc 10 from contracted configuration 20 into the expanded configuration 19 may be triggered or effected in at least one of several ways. For an elastic material such as stainless steel or titanium, the dynamic loaded intervertebral artificial disc 10 may be fabricated to be in an expanded configuration 19 at rest with no force applied. The application of a compressive force by a device such as an insertion tool may be used to alter, including reduce and expand, the effective height of intervertebral artificial disc 10.

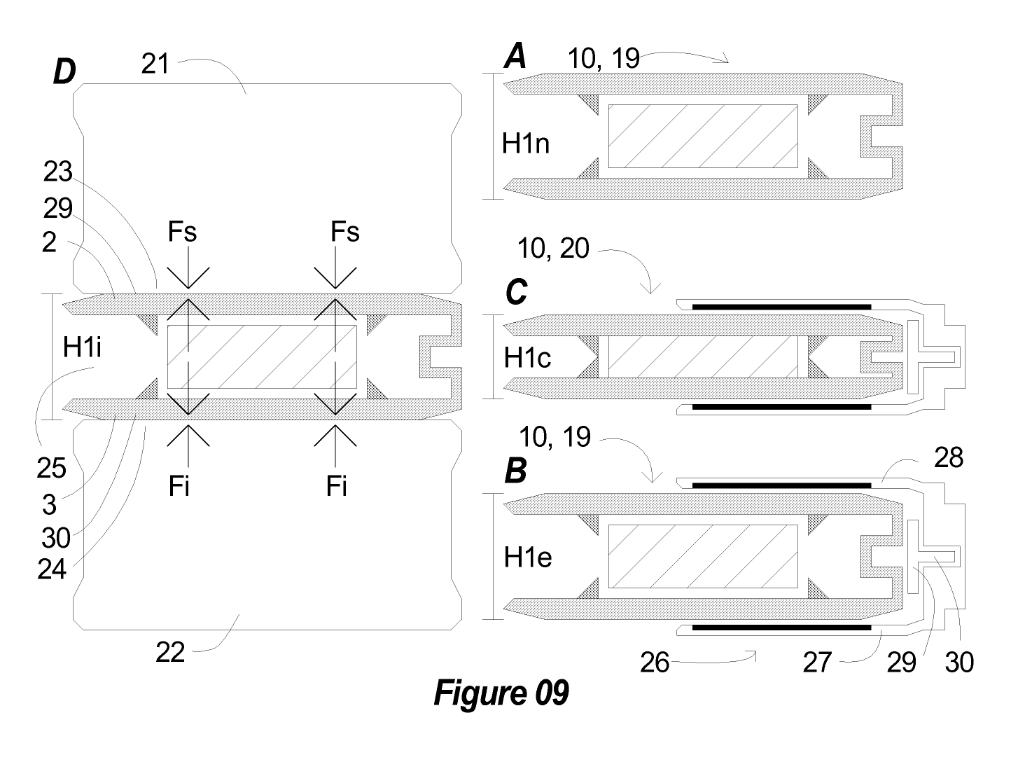

FIG. 9 depicts a dynamic loaded intervertebral artificial disc 10 in various forms before and after insertion into the intervertebral space 25. In one embodiment, dynamic loaded element 1 is fabricated from an elastic material, such as titanium or stainless steel or other biocompatible metal or material.

FIG. 9A shows a dynamic loaded intervertebral artificial disc 10 in the expanded position. No force is being applied in this figure, and the intervertebral artificial disc 10 is shown in its expanded position 19, with height H1e, which may also be its neutral position H1n, as shown (here H1e=H1n).

FIG. 9B shows a dynamic loaded intervertebral artificial disc 10 in the expanded position 19, being engaged by insertion tool 26. Insertion tool 26 is shown with insertion tool gripper arm 27 and 28 holding intervertebral artificial disc 10. Minimal force is being applied in this figure, and the intervertebral artificial disc 10 is shown in its expanded position 19, with height H1e, which in this embodiment may also be its neutral position as shown.

FIG. 9C shows a dynamic loaded intervertebral artificial disc 10 in the compressed position 20, being engaged by insertion tool 26. Insertion tool 26 is shown with insertion tool gripper arm 27 and 28 applying compressive force to intervertebral artificial disc 10. Sufficient force is being applied in this figure to compress intervertebral artificial disc 10 into a contracted configuration 20, with height H1c.

FIG. 9D shows a dynamic loaded intervertebral artificial disc 10 inserted into the intervertebral space 25 in between superior vertebral body 21 and inferior vertebral body 22. Superior endplate articulating surface 2 is shown in contact with and applying superior disc-vertebral body interface force Fs to inferior endplate 23 of superior vertebral body 21, which is in turn applying an equal and opposite force in response. Inferior endplate articulating surface 3 is shown in contact with and applying inferior disc-vertebral body interface force Fi to superior endplate 24 of inferior vertebral body 22, which is in turn applying an equal and opposite force in response.

The dynamic loaded intervertebral artificial disc 10 is shown implanted, with height H1i, which is typically at a value in between H1e and H1c or in between H1n and H1c, with the actual value dependent upon the magnitude of the applied and exerted endplate forces, superior disc-vertebral body interface force Fs and inferior disc-vertebral body interface force Fi, which when in steady state are generally approximately equal, though other forces may be applied which could cause these forces to be unequal.

The dynamic loaded intervertebral artificial disc 10 taught in FIG. 9 provides several significant advantages over existing devices for intervertebral disc replacement and fusion techniques and technologies:

(1) By facilitating a reduction in height, typically from H1e to H1c, during implantation, the insertion forces required are dramatically reduced or eliminated. Typically, in preparation for implantation of an artificial discs (for a vertebral arthroplasty procedure) and vertebral fusion cages (for a vertebral fusion procedure), the damaged intervertebral disc is removed and the vertebral endplates adjacent to the disc being replaced are filed down with a series of tools, often including a rasp. The purposes of this procedure are multifold: (A) to clean the endplate to facilitate improved fusion rates as well as (B) to create space and to shape the channel to match the typically generally rectangular cross section of the implanted artificial disc or fusion cage.

This procedure can damage the endplate, resulting in perforation of the endplate and weakening of the remaining vertebral body. One consequence is mechanical instability of the implanted artificial disc or fusion cage as well as possible subsidence (sinking of the implanted disc or cage into the vertebral body), with loss of height and collapse of the adjacent vertebral bodies into the disc space.

(2) Because the height of the implanted artificial disc or fusion cage is constant in existing technologies, a great deal of force is required to insert the implant into the intervertebral space 25. The implant is maintained in its position post-implantation by the surface friction between the inferior endplate 23 and superior endplate articulating surface 2 and the surface friction between the superior endplate 24 and inferior endplate articulating surface 3. This friction is approximately the sum of the superior disc-vertebral body interface force Fs multiplied by the coefficient of friction at this implant-vertebral body interface plus the inferior disc-vertebral body interface force Fi multiplied by the coefficient of friction at this implant vertebral body interface.

Since a high friction force is desired to maintain the implant in a stable position post-implantation, at least until bony fusion or bony in-growth into the implant articulating surface occurs, a high force is therefore necessarily required during the implantation procedure. These forces are usually applied through the use of a surgical mallet which is hit against a tamp which transmits the force impulse from the impact directly to the implant, advancing it along its course between the adjacent vertebral bodies. Although this is usually well-controlled by the surgeon, these forces can cause the implant to advance farther than desired. Depending on the trajectory of the implant being placed, such overshoot can result in neurological damage (by impinging on the spinal cord, neural ganglion, or spinal nerve root) or vascular damage (by impinging on the aorta, vena cave, iliac arteries, iliac veins, anterior segmental medullary arteries, posterior segmental medullary arteries, artery of Adamkiewicz, or other artery or vein.

The present invention facilitates the reduction in height H1 of the implant during implantation to a value H1c which may be less than the height of the intervertebral space 25, facilitating careful and precise insertion without the application of large forces or force impulses, thereby enhancing both the precision and the safety of the procedure.

(3) By applying a baseline compressive force to the adjacent vertebral body endplates (inferior endplate 23 and superior endplate 24), dynamic loaded intervertebral artificial disc 10 stimulates bone strengthening in this region of the according to Wolff's law. Superior endplate articulating surface 2 applies superior disc-vertebral body interface force Fs across superior interface 29 to inferior endplate 23 of superior vertebral body 21, stimulating bone growth along inferior endplate 23 and within superior vertebral body 21, thereby strengthening the construct. Inferior endplate articulating surface 3 applies inferior disc-vertebral body interface force Fi across inferior interface 30 to superior endplate 24 of inferior vertebral body 22, stimulating bone growth along superior endplate 24 and within inferior vertebral body 22, thereby strengthening the construct.

This technique with appropriately designed fusion cage and artificial disc may be used to strengthen the resulting spine and implant mechanics in a spinal fusion and for a spinal arthroplasty, respectively.

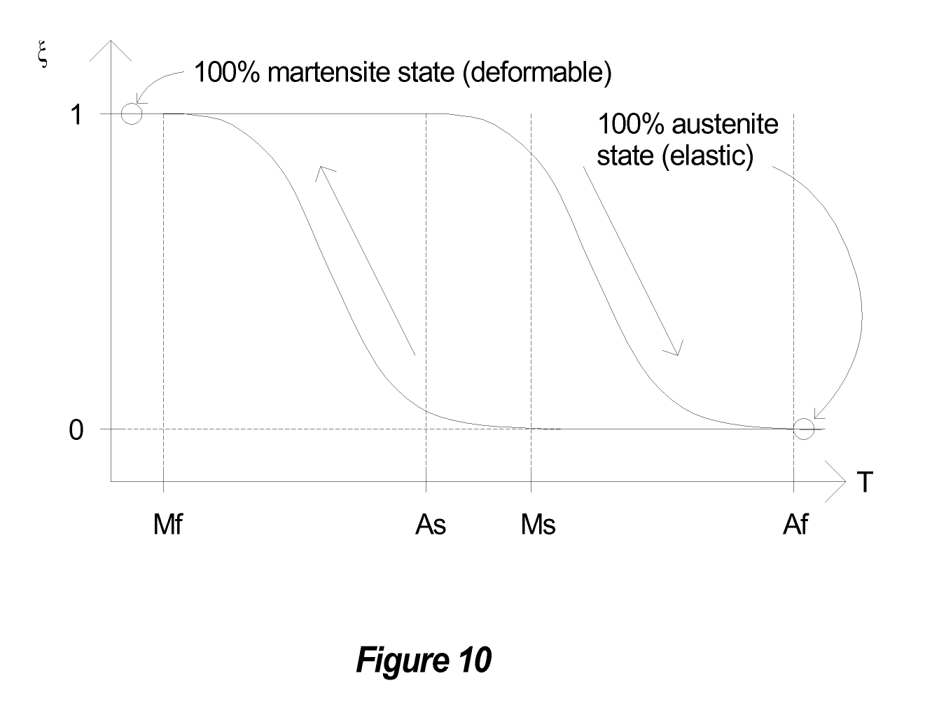

FIG. 10 depicts a temperature transition diagram for a shape memory material, such as a shape memory alloy, representative of those used in the present invention.

The horizontal axis represents temperature T, and the vertical axis represents the martensite fraction, the portion of the material which is in the martensite state, ranging fro 0 to 1. Cooling brings a greater proportion of the material into the martensite state, which exhibits greater deformability. Heating brings a greater proportion of the material into the austenite state, which exhibits greater rigidity elasticity which is useful in applying forces against bones or other structures as taught herein.

One preferred alloy which is biocompatible is nickel-titanium (NiTi, typically approximately 55% Nickel, though other proportions may be used), though others may be used without departing from the present invention. NiTi alloys change from austenite to martensite upon cooling; Ms and Mf are the temperatures at which the transition to martensite start and finish during cooling. Correspondingly, during heating As and Af are the temperatures at which the transformation from Martensite to Austenite start and finish. This is diagrammed below:

Cooling: austenite state→martensite state
(transition starts at temperature Ms and completes at temperature Mf)

Warming: martensite state→austenite state (complete at temperature Af)
(transition starts at temperature As and completes at temperature Af)

In the martensite state the alloy is typically more malleable and elastic.

dynamic loaded intervertebral artificial disc 10 in various forms before and after insertion into the intervertebral space 25. In one embodiment, dynamic loaded element 1 is fabricated from a shape memory alloy material. Such shape memory alloys include but are not limited to silver-cadmium (Ag—Cd), copper-aluminum-nickel (Cu—Al—Ni), copper-tin (Cu—Sn), copper-zinc (Cu—Zn), copper-zinc-silicon (Cu—Zn—Si), copper-zinc-aluminum (Cu—Zn—Al), copper-zinc-tin (Cu—Zn—Sn), iron-platinum (Fe—Pt), manganese-copper (Mn—Cu), iron-manganese-tin (Fe—Mn—Si), platinum alloys (Pt—X), cobalt-nickel-aluminum (Co—Ni—Al), cobalt-nickel-gallium (Co—Ni—Ga), nickel-iron-gallium (Ni—Fe—Ga), titanium-palladium (Ti—Pd), nickel-titanium (Ni—Ti), nickel-titanium-niobium (Ni—Ti—Nb), nickel-manganese-gallium (Ni—Mn—Ga), or other shape memory alloy or material having these or comparable or equivalent properties.

Furthermore, apparatus taught in the present invention may be fabricated from or comprising shape memory polymers, without departing from the present invention. Shape memory polymers (SMPs) may be triggered to change shape in response to temperature change, electric field, magnetic field, light, or solution.

Materials with shape memory polymers (SMP) characteristics include but are not limited to polyurethanes, polyurethanes with ionic or mesogenic components made by prepolymer method, block copolymer of polyethylene terephthalate (PET), block copolymer of polyethyleneoxide (PEO), block copolymers containing polystyrene and poly(1,4-butadiene), an ABA triblock copolymer made from poly(2-methyl-2-oxazoline) and polytetrahydrofuran, a linear amorphous polynorbornene, and an organic-inorganic hybrid polymers consisting of polynorbornene units that are partially substituted by polyhedral oligosilsesquioxane (POSS).

The term “shape memory alloys” is used herein to also comprise shape memory polymers, which may be used in addition air instead of shape memory alloys, depending on the material properties desired. The reversible (elastic) motion of domain boundaries during the phase transformation between the austenitic and martensitic phases produces pseudoelasticity (which may sometimes be called superelasticity). This is a characteristic exhibited by shape memory alloys

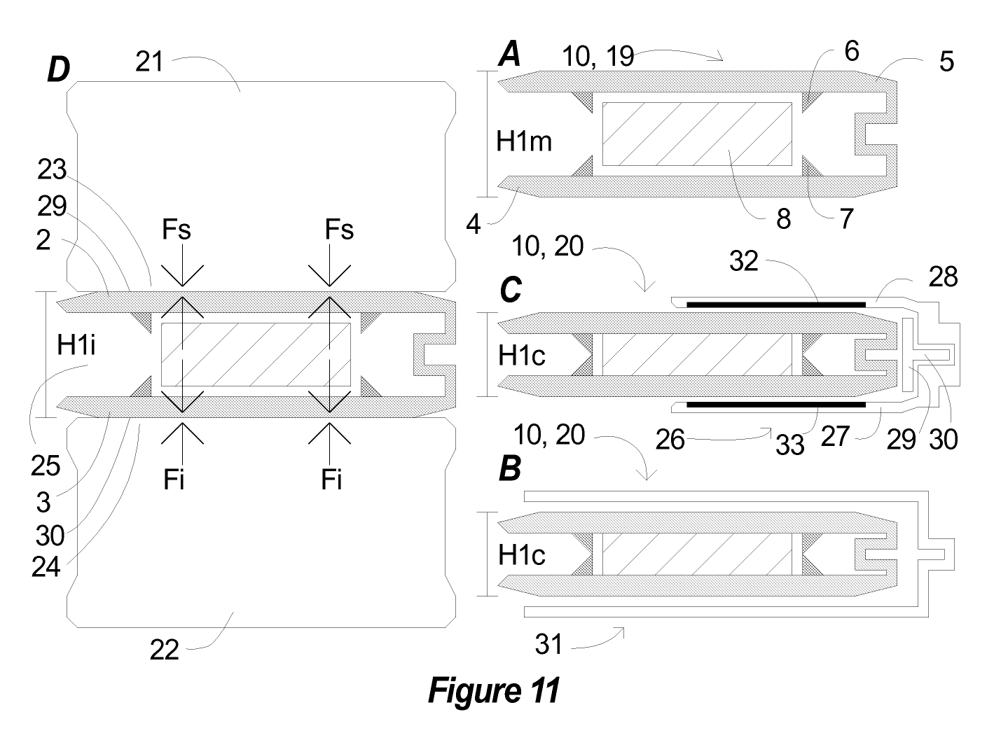

FIG. 11 depicts a dynamic loaded intervertebral artificial disc 10 in various forms before and after insertion into the intervertebral space 25. In one embodiment, dynamic loaded element 1 is fabricated from a shape memory alloy material, such as nickel-titanium or other material.

FIG. 11A shows a dynamic loaded intervertebral artificial disc 10 in the expanded position. No force is being applied in this figure, and the intervertebral artificial disc 10 is shown in its expanded position 19, with height H1m, shown to denote what may be the “memorized” position of the shape memory material. (here H1e=H1m).

FIG. 11B shows a dynamic loaded intervertebral artificial disc 10 in the contracted configuration 20. Dynamic loaded intervertebral artificial disc 10 is shown within temperature control chamber 31. The transition of in shape represented in the change from FIG. 11A to 11B may be facilitated by the application of a cold temperature by temperature control chamber 31. Such a cold temperature would effect a transition in temperature of dynamic loaded intervertebral artificial disc 10 down to and past Ms (start of transformation to martensite state) to or below Mf (finish of transformation to martensite state). In practice, temperature Mf is typically substantially below body temperature, and it may be maintained as shown by temperature control chamber 31 or by placement in a freezer or cooler with ice, dry ice, or other chilled material, active chilling device, passive chilling device, other cooling device, or combination thereof.

FIG. 11C shows a dynamic loaded intervertebral artificial disc 10 in the compressed position 20, being engaged by insertion tool 26. Insertion tool 26 is shown with insertion tool gripper arm 27 and 28 in contact with intervertebral artificial disc 10. Insertion tool gripper arm 27 and 28 may apply no force, minimal force, or substantial force to intervertebral artificial disc 10. Intervertebral artificial disc 10 is maintained at a height of H1c by temperature control features and function of insertion tool 26, which maintains the temperature of intervertebral artificial disc 10 at a sufficiently low value, usually at a temperature below Ms and below or approximately Mf; however, other temperatures, such as temperatures between Mf and Ms may also be used.

Insertion tool 26 comprises temperature control element 32 and 33, which may be attached to, contained within, recessed along, or otherwise associated or in contact with insertion tool gripper arm 27 and 28, respectively. Temperature control element 32 and 33 serve to control the temperature of intervertebral artificial disc 10 to maintain it in the desired temperature range and state, typically in a martensite state, providing maximal deformability to maintain the intervertebral artificial disc 10 in a contracted configuration 20 at a height of H1c, which is substantially less than the intervertebral disc space height H1i, thereby facilitating easy insertion without requiring large forces and the use of surgical mallets and tamps. To maintain the intervertebral artificial disc 10 in a martensite state, a temperature typically between or below Ms and Mf or preferably below Mf, if maximal effect is desired, is used.

Temperature control element 32 and 33 may be implemented in at least one of several ways, without departing from the present invention. In one embodiment, temperature control element 32 and 33 are implemented as tubes fabricated preferably from a thermally conductive material such as titanium, copper, aluminum, stainless steel, annealed pyrolytic graphite, through which a sufficiently cool cooling fluid passes. This cooling fluid may be chilled air, chilled water, liquid nitrogen, liquid oxygen, liquid helium, liquid hydrogen, other fluid material, other gaseous material, other liquid materials. In another preferred embodiment, temperature control element 32 and 33 comprise thermoelectric cooling elements which use the Peltier effect to create a thermal gradient, thereby cooling the intervertebral artificial disc 10, to below Ms and preferably to below Mf, driving the shape memory material components of the intervertebral artificial disc 10 into the martensite state, resulting in a reduction in the height of intervertebral artificial disc 10 to contracted height H1c. Yet another embodiment includes the use of synthetic jet and droplet atomization technologies to enhance heat transfer and removal.

While in the contracted configuration 20, intervertebral artificial disc 10 is inserted into intervertebral space 25. As discussed in the section for FIG. 9, there are multiple advantages provided by the capability to change the height of intervertebral artificial disc 10, and these are also afforded by the apparatus and method taught using shape memory materials in FIG. 11.

FIG. 11D shows a dynamic loaded intervertebral artificial disc 10 inserted into the intervertebral space 25 in between superior vertebral body 21 and inferior vertebral body 22. Superior endplate articulating surface 2 is shown in contact with and applying superior disc-vertebral body interface force Fs to inferior endplate 23 of superior vertebral body 21, which is in turn applying an equal and opposite force in response. Inferior endplate articulating surface 3 is shown in contact with and applying inferior disc-vertebral body interface force Fi to superior endplate 24 of inferior vertebral body 22, which is in turn applying an equal and opposite force in response.

The dynamic loaded intervertebral artificial disc 10 is shown implanted, with height H1i, which is typically at a value in between H1e and H1c or in between H1n and H1c, with the actual value dependent upon the magnitude of the applied and exerted endplate forces, superior disc-vertebral body interface force Fs and inferior disc-vertebral body interface force Fi, which when in steady state are generally approximately equal, though other forces may be applied which could cause these forces to be unequal.

The dynamic loaded intervertebral artificial disc 10 taught in FIG. 11 provides several significant advantages over existing devices for intervertebral disc replacement and fusion techniques and technologies:

(1) By facilitating a reduction in height, typically from H1e to H1c, during implantation, the insertion forces required are dramatically reduced or eliminated. Typically, in preparation for implantation of an artificial discs (for a vertebral arthroplasty procedure) and vertebral fusion cages (for a vertebral fusion procedure), the damaged intervertebral disc is removed and the vertebral endplates adjacent to the disc being replaced are filed down with a series of tools, often including a rasp. The purposes of this procedure are multifold: (A) to clean the endplate to facilitate improved fusion rates as well as (B) to create space and to shape the channel to match the typically generally rectangular cross section of the implanted artificial disc or fusion cage.

This procedure can damage the endplate, resulting in perforation of the endplate and weakening of the remaining vertebral body. One consequence is mechanical instability of the implanted artificial disc or fusion cage as well as possible subsidence (sinking of the implanted disc or cage into the vertebral body), with loss of height and collapse of the adjacent vertebral bodies into the disc space.

(2) Because the height of the implanted artificial disc or fusion cage is constant in existing technologies, a great deal of force is required to insert the implant into the intervertebral space 25. The implant is maintained in its position post-implantation by the surface friction between the inferior endplate 23 and superior endplate articulating surface 2 and the surface friction between the superior endplate 24 and inferior endplate articulating surface 3. This friction is approximately the sum of the superior disc-vertebral body interface force Fs multiplied by the coefficient of friction at this implant-vertebral body interface plus the inferior disc-vertebral body interface force Fi multiplied by the coefficient of friction at this implant vertebral body interface.

Since a high friction force is desired to maintain the implant in a stable position post-implantation, at least until bony fusion or bony in-growth into the implant articulating surface occurs, a high force is therefore necessarily required during the implantation procedure. These forces are usually applied through the use of a surgical mallet which is hit against a tamp which transmits the force impulse from the impact directly to the implant, advancing it along its course between the adjacent vertebral bodies. Although this is usually well-controlled by the surgeon, these forces can cause the implant to advance farther than desired. Depending on the trajectory of the implant being placed, such overshoot can result in neurological damage (by impinging on the spinal cord, neural ganglion, or spinal nerve root) or vascular damage (by impinging on the aorta, vena cave, iliac arteries, iliac veins, anterior segmental medullary arteries, posterior segmental medullary arteries, artery of Adamkiewicz, or other artery or vein.

The present invention facilitates the reduction in height H1 of the implant during implantation to a value H1c which may be less than the height of the intervertebral space 25, facilitating careful and precise insertion without the application of large forces or force impulses, thereby enhancing both the precision and the safety of the procedure.

(3) By applying a baseline compressive force to the adjacent vertebral body endplates (inferior endplate 23 and superior endplate 24), dynamic loaded intervertebral artificial disc 10 stimulates bone strengthening in this region of the according to Wolff's law. Superior endplate articulating surface 2 applies superior disc-vertebral body interface force Fs across superior interface 29 to inferior endplate 23 of superior vertebral body 21, stimulating bone growth along inferior endplate 23 and within superior vertebral body 21, thereby strengthening the construct. Inferior endplate articulating surface 3 applies inferior disc-vertebral body interface force Fi across inferior interface 30 to superior endplate 24 of inferior vertebral body 22, stimulating bone growth along superior endplate 24 and within inferior vertebral body 22, thereby strengthening the construct.

This technique with appropriately designed fusion cage and artificial disc may be used to strengthen the resulting spine and implant mechanics in a spinal fusion and for a spinal arthroplasty, respectively.

Dynamic loaded intervertebral artificial disc 10 and any permutation of its components including but not limited to: posterior elastic element 5, anterior elastic element 4, dynamic loaded element 1, superior endplate articulating surface 2, inferior endplate articulating surface 3, and viscoelastic insert block 8 may be constructed from a single or multiplicity of materials including but not limited to shape memory material, elastic material, polymer material, other material. Such shape memory materials include but are not limited to shape memory alloys (including but not limited to silver-cadmium (Ag—Cd), copper-aluminum-nickel (Cu—Al—Ni), copper-tin (Cu—Sn), copper-zinc (Cu—Zn), copper-zinc-silicon (Cu—Zn—Si), copper-zinc-aluminum (Cu—Zn—Al), copper-zinc-tin (Cu—Zn—Sn), iron-platinum (Fe—Pt), manganese-copper (Mn—Cu), iron-manganese-tin (Fe—Mn—Si), platinum alloys (Pt—X), cobalt-nickel-aluminum (Co—Ni—Al), cobalt-nickel-gallium (Co—Ni—Ga), nickel-iron-gallium (Ni—Fe—Ga), titanium-palladium (Ti—Pd), nickel-titanium (Ni—Ti), nickel-titanium-niobium (Ni—Ti—Nb), nickel-manganese-gallium (Ni—Mn—Ga), or other shape memory alloy or material having these or comparable or equivalent properties), shape memory polymers (include but are not limited to polyurethanes, polyurethanes with ionic or mesogenic components made by prepolymer method, block copolymer of polyethylene terephthalate (PET), block copolymer of polyethyleneoxide (PEO), block copolymers containing polystyrene and poly(1,4-butadiene), an ABA triblock copolymer made from poly(2-methyl-2-oxazoline) and polytetrahydrofuran, a linear amorphous polynorbornene, and an organic-inorganic hybrid polymers consisting of polynorbornene units that are partially substituted by polyhedral oligosilsesquioxane), and other shape memory materials or materials with substantially equivalent properties. Such elastic materials include but are not limited to titanium, stainless steel, nickel, other biocompatible metals, other metals, and other elastic materials. Such polymer materials include but are not limited to polyetheretherketone, polytetrafluoroethylene (PTFE, a synthetic fluoropolymer of tetrafluoroethylene, also known as Teflon).

Such other materials include but are not limited to synthetic polymers, thermoplastic elastomers, silicone elastomers, styrene block copolymers, thermoplastic copolyesters, thermoplastic polyamides, thermoplastic polyolefins, thermoplastic polyurethanes, thermoplastic vulcanizates, polyvinyl chloride, fluoropolymers, polytetrafluoroethylene (PTFE), modified polytetrafluoroethylene (modified PTFE), fluorinated ethylene-propylene (FEP), ethylene tetrafluoroethylene (ETFE), perfluoroalkoxy (PFA, or Teflon-PFA), perfluoroalkoxy (methyl vinyl ether) (MFA), polyurethane, polycarbonate, silicone, acrylic compounds, polypropylene, low density polyethylenes, nylon, sulfone resins, high density polyethylenes, natural polymers, cellulose polymers, collagen, other natural polymers, hyaluronic acid, alginates, carrageenan, biocompatible metals, precious metals, gold, silver, other precious metals, stainless steel, titanium, other biocompatible metals, biocompatible ceramics, porcelain, alumina, hydroxyapatite, zirconia, other ceramics & related materials, polyvinyl chloride, fluoropolymer, polyurethane, polycarbonate, silicone, acrylic, thermoplastic, polypropylene, low density polyethylenes, nylon, sulfone, high density polyethylenes, other synthetic biocompatible polymer, natural biocompatible polymer, cellulose polymer, collagen, starch blend, metal, precious metal, stainless steel, titanium, other biocompatible metal, ceramic, or other biocompatible material, or other material.

Implantation: Dynamic loaded intervertebral artificial disc 10 is either stored at a cold temperature, below Ms and preferably below Mf, or chilled to this temperature prior to implantation using temperature control chamber 31, or insertion tool 26 equipped with temperature control element 32 and 33, or using other temperature control means. This cooling process causes typically at least one of the posterior elastic element 5 and anterior elastic element 4, and may cause at least one of superior endplate articulating surface 2 and inferior endplate articulating surface 3, to partially or completely enter the martensite state, resulting in dynamic loaded intervertebral artificial disc 10 transitioning from an expanded configuration 19 to a contracted configuration 20, with a height changing from approximately H1e to approaching or approximately H1c. Dynamic loaded intervertebral artificial disc 10 is then inserted into intervertebral space 25. Dynamic loaded intervertebral artificial disc 10 is then warmed either passively using heat absorbed from the body or actively using any of several apparatus or methods including but not limited to temperature control element 32 and 33 on insertion tool 26. Temperature control element 32 and 33 may use any method or apparatus to perform this heating including but not limited to resistive electrical heating, flow of heated gas, flow of heated liquid, flow of heated fluid, thermoelectric heating (using such mechanism as the Peltier effect or other mechanism), or other method or apparatus to perform heating. This heating process, typically to above As and preferably to above or approximately Af, causes shape memory components of dynamic loaded intervertebral artificial disc 10 to transition from the martensite (in which ξ≅1 or ξ=1, as diagrammed in FIG. 10) state to the austenite state (in which ξ≅0 or ξ=0, as diagrammed in FIG. 10), thereby causing dynamic loaded intervertebral artificial disc 10 to expand from a contracted configuration 20 to an expanded configuration 19, resulting in a height increase from approximately H1c to H1i. H1i may typically be in between H1c and H1m, with H1i typically being determined by the preoperative height of intervertebral disc space 25 as well as the amount of disc and bone removed prior to implantation of dynamic loaded intervertebral artificial disc 10, as well as by the relative spring constants (an correspondingly their inverses, the compliances) of the dynamic loaded intervertebral artificial disc 10 and the relevant anatomical structures comprising the remaining intervertebral disc, intervertebral disc nucleus pulposus, intervertebral disc annulus, facet joints, anterior longitudinal ligament, posterior longitudinal ligament, ligamentum flavum, and other ligaments and muscles spanning the intervertebral disc space.

In one embodiment, the dynamic loaded element 1 is fabricated from a shape memory alloy, including but not limited to nickel-titanium. In an alternate embodiment, dynamic loaded element 1 is fabricated from a metal such as titanium or stainless steel or other biocompatible metal or material. Dynamic loaded element 1 may be constructed from at least one of stainless steel, titanium, polyetheretherketone, nickel-titanium alloy, other shape memory alloy, other material, and combination thereof. Viscoelastic insert block 8 may be constructed from at least one of silicone, Teflon, polyetheretherketone, other material listed above in this figure description, and other material. Viscoelastic insert block 8 may be omitted from dynamic loaded intervertebral artificial disc 10, without departing form the present invention.

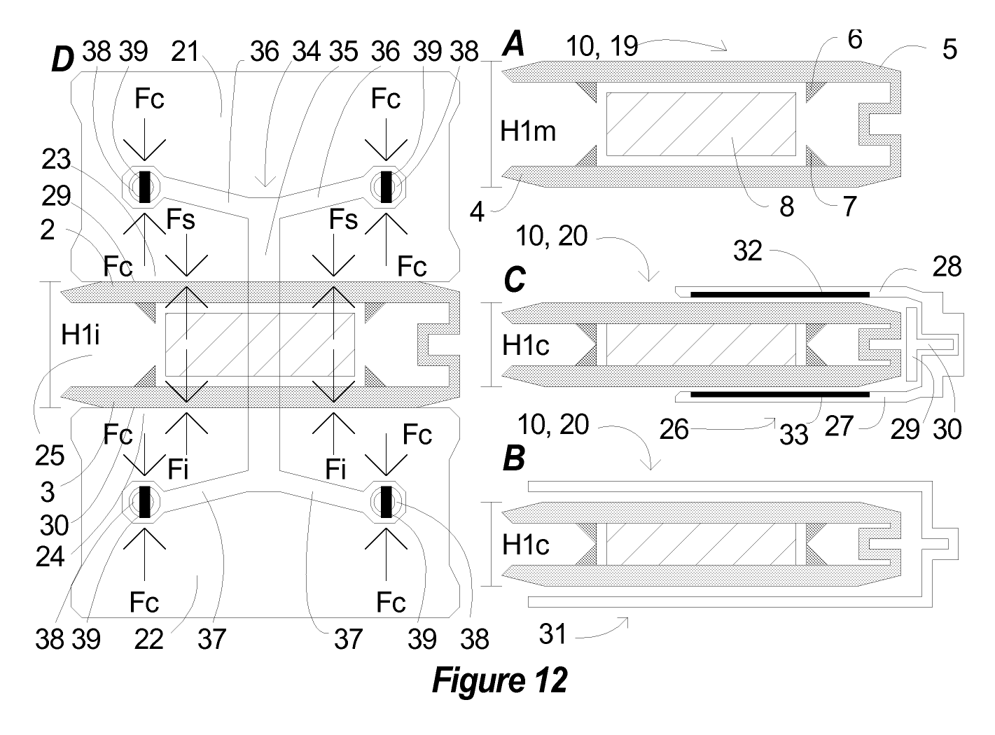

FIG. 12 depicts a dynamic loaded intervertebral artificial disc 10, as shown in FIGS. 1, 2, 3, 4, 5, 6, 7, 8, 9, and 11, with the addition of dynamic loaded compression plate 34, which may be configured to exert a compressive force across intervertebral disc space, to accelerate spinal fusion in the case of placement of a fusion cage and to enhance bony ingrowth and mechanical stabilization across the implant bone interfaces (superior interface 29 and inferior interface 30) in the case of a vertebral arthroplasty. Alternatively, dynamic loaded compression plate 34 may be configured to exert at least one of a compression force, a tension force, a neutral force, a stabilizing force, and a force vector driving the vertebral bodies toward a target distance apart or together, without departing from the present invention.

Dynamic loaded compression plate 34 comprises a main strut 35, a single or multiplicity of superior dynamic loaded arm 36, and a single or multiplicity of inferior dynamic loaded arm 37. In a preferred embodiment, each superior dynamic loaded arm 36 has at least one bone screw hole 38, configured to allow the placement of at least one bone screw 39 through the bone screw hole 38 and into superior vertebral body 21, and preferably through at least one cortical bone layer, typically also into the cancellous bone layer, and optionally into the cortical bone layer on the opposite side of superior vertebral body 21, for additional mechanical strength. In a preferred embodiment, each inferior dynamic loaded arm 37 has at least one bone screw hole 38, configured to allow the placement of at least one bone screw 39 through the bone screw hole 38 and into inferior vertebral body 22, and preferably through at least one cortical bone layer, typically also into the cancellous bone layer, and optionally into the cortical bone layer on the opposite side of superior vertebral body 21, for additional mechanical strength.

The construct shown in FIG. 12, as well as all others taught in the present invention, may be used on any vertebral level, including occipitocervical, cervical, cervicothoracic, thoracic, thoracolumbar, lumbar, lumbosacral, and sacrococcygeal. The construct shown in FIG. 12, as well as all others taught in the present invention, may be used for any or all surgical approaches, including anterior, posterior, lateral, and variations and combinations of theses.

Such specific procedures to which the present invention has applicability include but are not limited to anterior cervical discectomy and fusion (ACDF), anterior cervical discectomy and arthroplasty, anterior cervical fusion (ACF), lateral cervical discectomy and fusion, lateral cervical fusion, lateral cervical discectomy with arthroplasty, posterior cervical instrumentation, posterior cervical discectomy and fusion, posterior cervical discectomy and arthroplasty, and other cervical procedures.

Such specific procedures to which the present invention also has applicability include but are not limited to anterior thoracic discectomy and fusion, anterior thoracic fusion, posterolateral thoracic discectomy and fusion, posterolateral thoracic fusion, posterior thoracic instrumentation, posterior thoracic discectomy and fusion, posterior thoracic discectomy and arthroplasty, and other thoracic procedures.

Such specific procedures to which the present invention also has applicability include but are not limited to anterior lumbar discectomy and fusion, anterior lumbar Interbody fusion (ALIF), anterior lumbar fusion, posterolateral lumbar discectomy and fusion, posterolateral lumbar fusion, extreme lateral lumbar discectomy and fusion (XLIF), retroperitoneal lumbar discectomy and fusion, posterior lumbar instrumentation, posterior lumbar discectomy and fusion, posterior lumbar discectomy and arthroplasty, posterior lumbar interbody fusion (PLIF), posterior lumbar interbody arthroplasty, transforaminal lumbar interbody fusion (TLIF), transforaminal lumbar interbody arthroplasty, and other lumbar procedures.

Such specific procedures to which the present invention also has applicability include but are not limited to anterior lumbosacral discectomy and fusion, anterior lumbosacral Interbody fusion, anterior lumbosacral fusion, posterolateral lumbosacral discectomy and fusion, posterolateral lumbosacral fusion, extreme lateral lumbosacral discectomy and fusion, retroperitoneal lumbosacral discectomy and fusion, posterior lumbosacral instrumentation, posterior lumbosacral discectomy and fusion, posterior lumbosacral discectomy and arthroplasty, posterior lumbosacral interbody fusion, posterior lumbosacral interbody arthroplasty, transforaminal lumbosacral interbody fusion, transforaminal lumbosacral interbody arthroplasty, and other lumbosacral procedures.

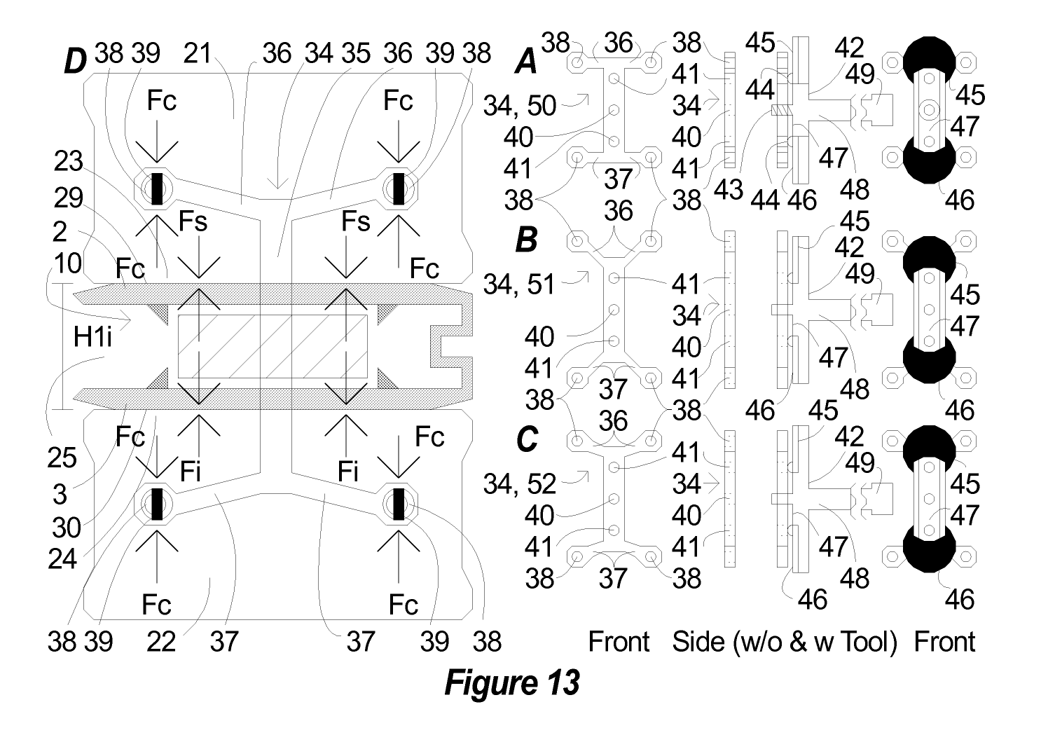

FIG. 13 depicts a dynamic loaded intervertebral artificial disc 10, as shown in FIGS. 1, 2, 3, 4, 5, 6, 7, 8, 9, and 11, with the addition of dynamic loaded compression plate 34, as also shown in FIG. 12, and which may be configured to exert a compressive force across intervertebral disc space, to accelerate spinal fusion in the case of placement of a fusion cage and to enhance bony ingrowth and mechanical stabilization across the implant bone interfaces (superior interface 29 and inferior interface 30) in the case of a vertebral arthroplasty. Alternatively, dynamic loaded compression plate 34 may be configured to exert at least one of a compression force, a tension force, a neutral force, a stabilizing force, and a force vector driving the vertebral bodies toward a target distance apart or together, without departing from the present invention.

Alternatively, dynamic loaded compression plate 34 may be used alone with the natural or diseased disc in place in order to encourage fusion or stabilization without a discectomy procedure. Alternatively, dynamic loaded compression plate 34 may be used alone with a discectomy procedure to enhance fusion of end plates to each other, i.e. inferior endplate 23 of superior vertebral body 21 to superior endplate 24 of inferior vertebral body 22, in order to encourage fusion and/or stabilization of the vertebral bodies or construct during the fusion process. Alternatively, dynamic loaded compression plate 34 may be used with a discectomy procedure and intervertebral graft placement to enhance fusion of end plates to the intervertebral graft, i.e. inferior endplate 23 of superior vertebral body 21 to superior portion of intervertebral graft and superior endplate 24 of inferior vertebral body 22 to inferior portion of intervertebral graft, in order to encourage fusion and/or stabilization of the vertebral bodies or construct during the fusion process.

FIG. 13A through 13C show methods, apparatus, surgical technique, and surgical tools taught for the implantation of dynamic loaded compression plate 34. On the left in each of these figures, a front view of dynamic loaded compression plate 34 is presented. In the center two images, a lateral view of dynamic loaded compression plate 34 is shown, with the left image showing the dynamic loaded compression plate 34 alone and the right image showing dynamic loaded compression plate 34 while being held by dynamic loaded compression plate placement tool 42. On the left in each of these figures, a front view of dynamic loaded compression plate 34 while being held by dynamic loaded compression plate placement tool 42 is presented.

The orientations may be in any body plane. For instance, for an anterior cervical plate, “front” corresponds to an antero-posterior view, while for a lateral thoracic plate or lateral lumbosacral plate, “front” corresponds to a lateral view.

Components and features of dynamic loaded compression plate 34 are described in more detail in FIG. 12.

FIG. 13A shows dynamic loaded compression plate 34 in it's “memory” configuration, termed dynamic plate memory configuration 50. Dynamic loaded compression plate 34 is attached to dynamic loaded compression plate placement tool 42, as shown on the right of this figure. Mounting screw 43 of dynamic loaded compression plate placement tool 42 is advanced into center guide hole 40 of dynamic loaded compression plate 34. Center guide hole 40 may be threaded to accept a thread on mounting screw 43. Alternatively, a spring loaded locking mechanism may be used to allow a variation or functional equivalent or alternate design of mounting screw 43 to be snapped into center guide hole 40. Alignment ridge 44 of dynamic loaded compression plate placement tool 42 is advanced into adjacent guide hole 41 of dynamic loaded compression plate 34. Adjacent guide hole 41 may be threaded to accept a thread on a variation of alignment ridge 44. Alternatively, a spring loaded locking mechanism may be used to allow a variation of alignment ridge 44 to be snapped into adjacent guide hole 41. Other apparatus or methods may be used to attach dynamic loaded compression plate 34 to dynamic loaded compression plate placement tool 42, without departing from the present invention.

Temperature control element 45 and 46, shown mounted onto, recessed within, or as a component of dynamic loaded compression plate placement tool 42 are used to control the temperature of dynamic loaded compression plate 34. Temperature control element 45 and 46, may be in thermal communication with any single or multiplicity of parts of dynamic loaded compression plate 34. In one embodiment, temperature control element 45 and 46 are shown in thermal communication with superior dynamic loaded arm 36 and inferior dynamic loaded arm 37, respectively, though the correspondence or alignment of these may be altered without departing from the preset invention. Placement tool shaft 48 extends from or is in mechanical communication with placement tool face 47 and is in mechanical communication with placement tool handle 49 for gripping by the surgeon or surgical assistant or other personnel.

FIG. 13B shows dynamic loaded compression plate 34 in it's “pre-loaded” configuration, termed dynamic plate pre-loaded configuration 51. Dynamic loaded compression plate 34 is attached to dynamic loaded compression plate placement tool 42, as shown on the right of this figure.

Transition of dynamic loaded compression plate 34 into dynamic plate pre-loaded configuration 51 may be achieved by any of several methods and apparatus without departing from the present invention. One preferred embodiment, as shown in FIG. 13 comprises the use of temperature control element 45 and 46 or any functional equivalent to transition or to maintain the temperature of dynamic loaded compression plate 34 to below Ms (as diagrammed in FIG. 10) or preferably to below Mf (as diagrammed in FIG. 10). In doing so, dynamic loaded compression plate 34 is driven into a martensite state, and as such exhibits enhanced deformability and elasticity, allowing for shaping by any one of several techniques, methods, and apparatus: (1) manually by the surgeon or assistant; (2) by struts, arms, levers, catches, clips, functional equivalents, or other components of the placement too; dynamic loaded compression plate placement tool 42; (3) by a tool which is separate from, accompanying, or associated with dynamic loaded compression plate placement tool 42, which facilitates the deformation of a single or multiplicity of components of dynamic loaded compression plate 34; and (4) by a tool which is separate from, accompanying, or associated with dynamic loaded compression plate placement tool 42, which facilitates the deformation of a single or multiplicity of at least one of main strut 35, superior dynamic loaded arm 36, inferior dynamic loaded arm 37, and other component of dynamic loaded compression plate 34.

In another preferred embodiment, dynamic loaded compression plate 34 may be deformed into dynamic plate pre-loaded configuration 51 and packaged and stored in this configuration as part of manufacturing process, preoperatively, or preoperatively, and maintained in this configuration with appropriate mechanical stabilizing materials. By this method, dynamic loaded compression plate 34 can be loaded onto dynamic loaded compression plate placement tool 42 and implanted in dynamic plate pre-loaded configuration 51, without requiring the additional step of deformation from dynamic plate memory configuration 50 into dynamic plate pre-loaded configuration 51, preoperatively or preoperatively.

In one preferred embodiment, dynamic loaded compression plate 34 is implanted while in dynamic plate pre-loaded configuration 51 and mechanically secured by the placement of a single or more typically a multiplicity of bone screws 39 inserted via respective bone screw hole 38 into superior vertebral body 21 and inferior vertebral body 22. Additionally, a single or multiplicity of bone screws 39 may be inserted via at least one of center guide hole 40 and adjacent guide hole 41 into intervertebral graft 53 or dynamic loaded intervertebral artificial disc 10, thereby providing additional mechanical stabilization of the construct and reducing the likelihood of movement or of retropulsion of intervertebral graft 53 or dynamic loaded intervertebral artificial disc 10 into spinal canal or other undesirable location.

FIG. 13C shows dynamic loaded compression plate 34 in it's “intermediate” configuration, termed dynamic plate intermediate configuration 52. This is representative of the configuration of dynamic loaded compression plate 34 after implantation and potentially after having exerted its compressive force Fc to enhance fusion (such as with intervertebral graft 53 or other fusion material or using other technique) or of incorporation of bone into the surface of an artificial disc such as dynamic loaded intervertebral artificial disc 10. The configuration of dynamic loaded compression plate 34, in dynamic plate intermediate configuration 52, is typically in between dynamic plate memory configuration 50 and dynamic plate pre-loaded configuration 51, though it could be approximately equal or similar to either of these or be outside of this range.

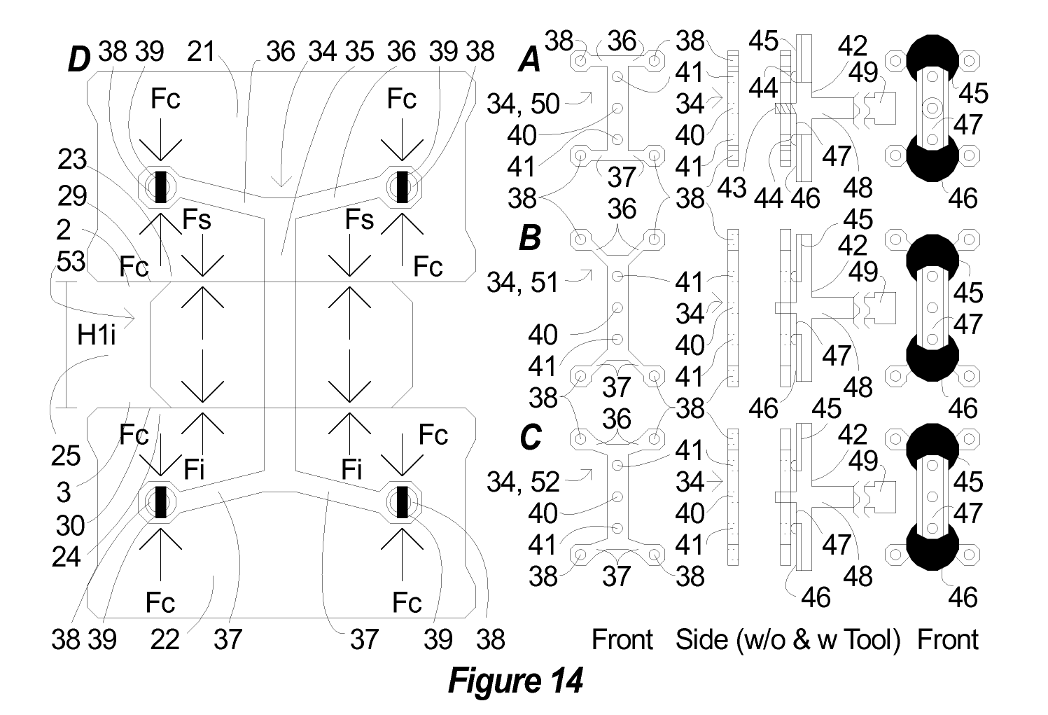

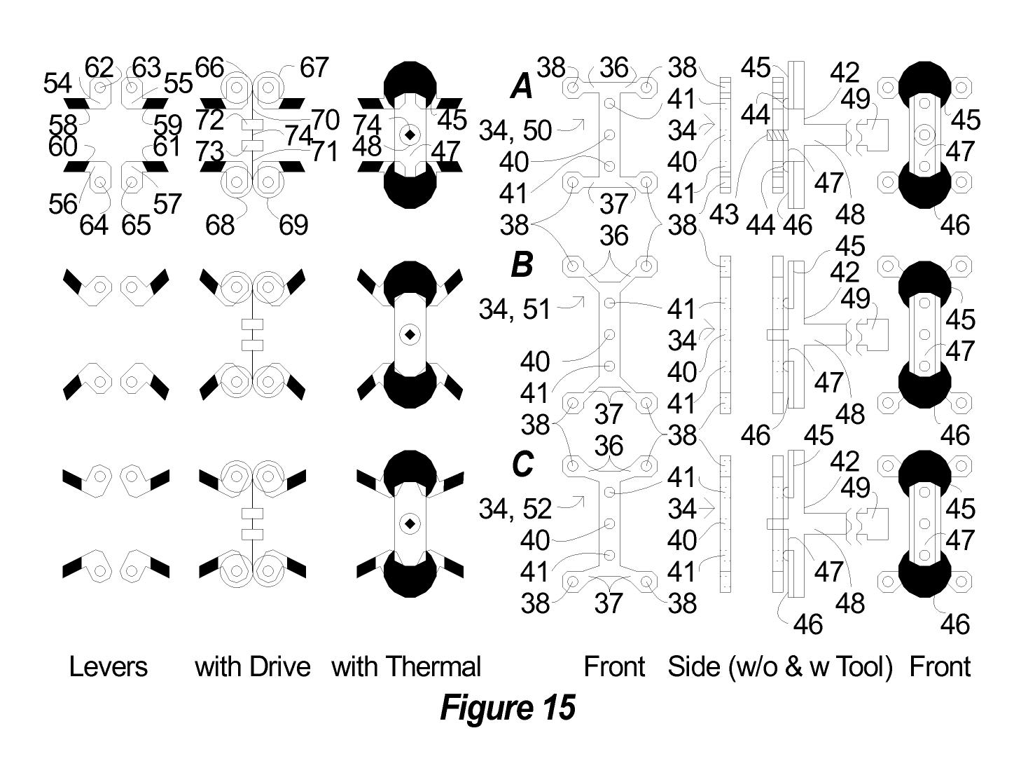

FIG. 14 depicts an intervertebral graft 53, instead of a dynamic loaded intervertebral artificial disc 10 as shown in FIG. 13, and is otherwise similar to FIGS. 1, 2, 3, 4, 5, 6, 7, 8, 9, and 11, with the addition of dynamic loaded compression plate 34, as also shown in FIG. 12, and which may be configured to exert a compressive force across intervertebral disc space, to accelerate spinal fusion in the case of placement of a fusion cage and to enhance bony ingrowth and mechanical stabilization across the implant bone interfaces (superior interface 29 and inferior interface 30) in the case of a vertebral arthroplasty. Alternatively, dynamic loaded compression plate 34 may be configured to exert at least one of a compression force, a tension force, a neutral force, a stabilizing force, and a force vector driving the vertebral bodies toward a target distance apart or together, without departing from the present invention.

FIG. 15 depicts the dynamic loaded compression plate 34 and dynamic loaded compression plate placement tool 42, as shown in FIGS. 13 and 14, with additional detail teaching one preferred embodiment for dynamic loaded compression plate placement tool 42. Additional embodiments for dynamic loaded compression plate placement tool 42 may become apparent to one skilled in the art, and these alternate or functionally equivalent or similar implementations do not depart from the present invention.

The left most column labeled “Levers” shows dynamic arm positioning levers 54, 55, 56, and 57 alone with respective dynamic arm positioning contacts 58, 59, 60, and 61, for clarity of view. The second column from the left, labeled “with Drive” further shows lever pulleys 66, 67, 68, and 69 as well as one preferred drive mechanism comprising drive cables 70 and 71, drive pulleys 72 and 73, and common drive cable 74. the third column form the left further details temperature control elements 45 and 46 along with placement tool face 47, common drive cable 74, and placement tool shaft 48. the right three columns are as described in FIGS. 13 and 14 and are reproduced for ease of comparison of the positions of elements comprising dynamic loaded compression plate placement tool 42 with deformations in dynamic loaded compression plate 34.

Dynamic arm positioning levers 54, 55, 56, and 57 apply at least one of a force, a torque, a linear displacement, and angular displacement via dynamic arm positioning contacts 58, 59, 60, and 61, respectively, to superior dynamic loaded arms 36 and 36 and inferior dynamic loaded arms 37 and 37, respectively, displacing said dynamic loaded arms from one position to another. One preferred such displacement is from dynamic plate memory configuration 50 to dynamic plate pre-loaded configuration 51. Alternatively, another preferred displacement from dynamic plate memory configuration 50 to dynamic plate intermediate configuration 52 may be accomplished. Displacements of differing magnitudes or polarity are also encompassed in the present invention. Furthermore, displacements involving differing magnitudes and/or differing polarities, or combinations thereof among the different Dynamic arm positioning levers 54, 55, 56, and 57 are also encompassed in the present invention.

Dynamic arm positioning contacts 58, 59, 60, and 61 may be formed from, attached to, bonded to, or otherwise may be in mechanical communication with dynamic arm positioning levers 54, 55, 56, and 57, respectively. Dynamic arm positioning contacts 58, 59, 60, and 61 may be thermally conductive to further facilitate temperature control of dynamic arm positioning levers 54, 55, 56, and 57 by dynamic loaded compression plate placement tool 42. Dynamic arm positioning contacts 58, 59, 60, and 61 may be thermally insulating to further facilitate more focal temperature control of dynamic arm positioning levers 54, 55, 56, and 57 by dynamic loaded compression plate placement tool 42 temperature control elements 45 and 46. Alternatively or in conjunction with temperature control elements 45 and 46, Dynamic arm positioning contacts 58, 59, 60, and 61 may have thermal actuator elements properties similar to or differing from temperature control elements 45 and 46, including Peltier effect properties, which may facilitate at lest one of more diffuse, more complex, and more uniform thermal patterning and/or control of the temperature of components of dynamic loaded compression plate 34, included but not limited to superior dynamic loaded arm 36 and inferior dynamic loaded arm 37.

Dynamic arm positioning levers 54, 55, 56, and 57 pivot around dynamic arm positioning hinges 62, 63, 64, and 65, respectively. Lever pulleys 66, 67, 68, and 69 are mechanically attached via any of several means to dynamic arm positioning levers 54, 55, 56, and 57, respectively, and transmit at least one of forces of displacements from drive cables 70 and 71. Drive cables 70 and 71 course around drive pulleys 72 and 73, respectively, and then at least one of connect with, join, are formed from, or otherwise transmit forces or displacements from common drive cable 74. Common drive cable 74 in turn is in mechanical communication with at least one of an actuator, a manual trigger, or other mechanism which provides at least one of a force, torque, linear displacement, angular displacement, or other for of force and/or energy.

One such preferred embodiment comprises a manual trigger pulled and/or pushed by the operator (i.e. surgeon) which exerts a tension and displacement on common drive cable 74, which through the apparatus and methods taught herein, effects movement of superior dynamic loaded arm 36 and 36 and inferior dynamic loaded arm 37 and 37, transitioning dynamic loaded compression plate 34 into dynamic plate pre-loaded configuration 51. Following this transition, dynamic loaded compression plate 34 may then be attached to vertebral bodies using bone screws as taught in FIGS. 12, 13, and 14.

Apparatus, methods, surgical tools and techniques of the present invention may also be used to fuse components of bones, such as pedicle fracture, spinous process fractures, facet fractures, and other vertebral, spinous, calvarum, and craniofacial fractures or for fusion of separate bones across suture lines.

The skilled practitioner may also envision application of these methods, apparatus, technologies, techniques, and surgical tools to other applications without departing from the present invention.

### CONCLUSION

It will be appreciated by those skilled in the art that while the invention has been described above in connection with the particular embodiments and examples, the invention is not necessarily so limited, and that numerous other embodiments, examples uses, modifications, and departures from the embodiments, examples, and uses are intended to be encompassed by the claims attached hereto The entire disclosure of each patent and publication cited herein is incorporated by reference, as if each such patent or publication were individually incorporated by reference herein.

We claim:

1. A device for providing enhanced bone fusion comprising:
A. A main strut; and
B. At least one dynamic loaded arm in mechanical communication with the main strut.

2. The device as in claim 1, comprising a single dynamic loaded arm.

3. The device as in claim 2, further comprising a second dynamic loaded arm.

4. The device as in claim 3, further comprising a third dynamic loaded arm.

5. The device as in claim 4, further comprising a fourth dynamic loaded arm.

6. The device as in claim 1, comprising a multiplicity of dynamic loaded arms.

6. The device as in claim 1, wherein the at least one dynamic loaded arm comprises”
A. A single or plurality of superior dynamic loaded arms; and
B. A single or plurality of inferior dynamic loaded arms.

7. The device as in claim 1, wherein the at least one dynamic loaded arm comprises”
A. Two superior dynamic loaded arms; and
B. Two inferior dynamic loaded arms.

8. The device as in claim 1, wherein at least one dynamic loaded arm comprises a shape memory alloy.

9. The device as in claim 8, wherein at least one dynamic loaded arm comprises a Nickel and Titanium alloy.

10. The device as in claim 9, wherein at least one dynamic loaded arm comprises a Nickel and Titanium alloy, wherein approximately 40 to 70% of the alloy is Titanium).

11. The device as in claim 1, wherein at least one dynamic loaded arm comprises a metal.

12. The device as in claim 1, wherein at least one dynamic loaded arm comprises a plastic.

13. A method for providing enhanced bone fusion comprising:
A. Formation of a dynamic loaded compression plate;
B. Deformation of said dynamic loaded compression plate; and
C. Attachment of said dynamic loaded compression plate to at least two vertebral bodies; and

14. The method of claim 13 wherein deformation of said dynamic loaded compression plate comprises elastic deformation.

15. The method of claim 14 wherein deformation of said dynamic loaded compression plate comprises elastic deformation of a metal.

16. The method of claim 13 wherein deformation of said dynamic loaded compression plate comprises pseudoelastic deformation.

17. The method of claim 13 wherein deformation of said dynamic loaded compression plate comprises deformation of a shape memory alloy.

18. The method of claim 13 further comprising altering the temperature of said dynamic loaded compression plate.

19. A surgical tool for implanting a device which provides enhanced bone fusion comprising:
A. A mounting screw;
B. A temperature control element; and
C. At least one positioning lever.

20. The surgical tool as in claim 19, further comprising a means for transmitting force from a trigger to the at least one positioning lever.
